(19) United States
(12) Patent Application Publication
(10) Pub. No.: US 2025/0121375 A1
(43) Pub. Date: Apr. 17, 2025

(54) PCR SYSTEM

(71) Applicant: Hewlett-Packard Development Company, L.P., Spring, TX (US)

(72) Inventors: Michael Cumbie, Corvallis, OR (US); Viktor Shkolnikov, Palo Alto, CA (US); Brian J. Keefe, San Diego, CA (US)

(73) Assignee: Hewlett-Packard Development Company, L.P., Spring, TX (US)

(21) Appl. No.: 18/702,194

(22) PCT Filed: Oct. 22, 2021
(86) PCT No.: PCT/US2021/056133

§ 371 Date: Apr. 17, 2024

(51) Int. Cl. B01L 7/00; B01L 3/00

(52) U.S. Cl. CPC B01L 7/52; B01L 3/502715; B01L 2300/0663; B01L 2300/0883; B01L 2300/1805; B01L 2300/1894; B01L 2400/0406

(57) ABSTRACT

A PCR system is described. The PCR system comprises a microfluidic cartridge having a thermocycling chamber, wherein a floor of the thermocycling chamber is substantially planar and is provided with a heater; a cooling module configured to engage with and be in thermal contact with a surface of the microfluidic cartridge; and an optical sensor configured to obtain optical signals from the thermocycling chamber. A method of performing PCR is also described.

### BACKGROUND

The Polymerase Chain Reaction (PCR) is used to amplify specific nucleic acid sequences and detect their presence in a sample. PCR can be used for many different applications, including quantification of gene expression, patient genotyping and also as a diagnostic tool to identify the presence of one or more pathogens, for example bacteria or viruses in a sample from a patient by amplifying and detecting nucleic acid sequences that are specific to a particular pathogen. Personalised medicine requires genotyping using PCR in which the detection of one or more biomarkers, for example specific mutations, may influence clinical decisions on the nature or type of medical intervention.

PCR subjects a sample to amplification conditions in the presence of an enzyme capable of elongating nucleic acid strands, for example a polymerase. The three basic steps of a single round or cycle of PCR amplification are denaturation, annealing and chain extension, each optimally taking place at different temperatures (in the range of 94-98° C. for denaturation; 50-65° C. for annealing, and 70-80° C. for chain extension, depending on polymerase), with each set of three steps being known by the term “thermocycling”. The amplification products (amplicons) are detected optically, for example using fluorescent reporters.

Microfluidic devices are used to transport, separate, mix or process fluids on a microscale. Microfluidic devices comprise a pattern of connected moulded or engraved microchannels which are incorporated into a microfluidic chip. Microfluidic devices can also be used to perform multi-step reactions such as PCR and are often referred to as “lab on chip” devices.

### BRIEF DESCRIPTION OF THE DRAWINGS

FIG. 1 is a schematic of an example PCR system;

FIG. 2 shows an example microfluidic cartridge;

FIG. 3 shows an example microfluidic cartridge with capillary breaks;

FIG. 4 shows a sequence of filling of a thermocycling chamber;

FIG. 5 shows an example cooling module;

FIG. 6 shows an example cooling module;

FIGS. 7A and 7B show an example cooling module from above and below;

FIG. 7C shows the cooling module of FIGS. 7A and 7B in thermal contact with a microfluidic cartridge; and

FIG. 8 shows an experimentally determined plot of heating and cooling rates for an example PCR system.

### DETAILED DESCRIPTION

Before particular embodiments of the present method and other aspects are disclosed and described, it is to be understood that the present method and other aspects are not limited to the particular process and materials disclosed herein as such may vary to some degree. It is also to be understood that the terminology used herein is used for the purpose of describing particular embodiments only and is not intended to be limiting, as the scope of the present method and other aspects will be defined only by the appended claims and equivalents thereof.

In the present specification, and in the appended claims, the following terminology will be used:
The singular forms “a”, “an” and “the” include plural referents unless the context clearly dictates otherwise. Thus, for example, reference to “a sensor” includes reference to one or more of such sensors.
The terms “about” and “approximately” when referring to a numerical value or range is intended to encompass the values resulting from experimental error that can occur when taking and/or making measurements.

Concentrations, amounts, and other numerical data may be presented herein in a range format. It is to be understood that such a range format is used merely for convenience and brevity and should be interpreted flexibly to include not only the numerical values explicitly recited as the limits of the range, but also to include all the individual numerical values or sub-ranges encompassed within that range as if each numerical value and sub-range is explicitly recited. For example, a weight range of approximately 1 wt. % to approximately 20 wt. % should be interpreted to include not only the explicitly recited concentration limits of 1 wt. % to approximately 20 wt. %, but also to include individual concentrations such as 2 wt. %, 3 wt. %, 4 wt. %, and sub-ranges such as 5 wt. % to 10wt. %, 10 wt. % to 20 wt. %, etc.

In the following description, for purposes of explanation, numerous specific details are set forth in order to provide a thorough understanding of the present apparatus and methods. It will be apparent, however, to one skilled in the art, that the present apparatus and methods maybe practiced without these specific details. Reference in the specification to “one example” or “an example” means that a particular feature, structure, or characteristic described in connection with the example is included in at least one example. The appearances of the phrase “in one example” in various places in the specification are not necessarily all referring to the same example.

Unless otherwise stated, any feature described herein can be combined with any aspect or any other feature described herein.

As used herein, the abbreviations “PCR”, “dNTPs” and “primers” refer to the “Polymerase Chain Reaction” and its components. Specifically, the term “dNTP” refers to the 2′-deoxynucleotide triphosphates used in PCR. The four standard dNTPs are 2′-deoxyadenosine 5′-triphosphate, 2′-deoxyguanosine 5′-triphosphate, 2′-deoxycytosine 5′-triphosphate and thymidine 5′-triphosphate (already lacking a 2′-hydroxyl), though modified dNTPs, for example non-natural nucleotides incorporating labels or reporter molecules, or reactive moieties may also be used (for example in the form of nucleobase modifications such as C7-modified deaza-guanine or C7-modified deaza-adenine or C5-modified cytosine or C5-modified thymidine). Non-natural nucleotides having for example sugar modifications (such as 2′-F or 2′-OMe modifications, or “LNA”, having an O—CH₂ linker between the 2′ and 4′ positions on the sugar), or backbone modifications such as phosphorothioates, or phosphorothiolates may also be used, as may unnatural bases such as the pyrimidine analog 6-amino-5-nitro-3-(1′-β-D-2′-deoxyribofuranosyl)-2(1H)-pyridone (dZ) and its Watson-Crick complement, the purine analog 2-amino-8-(1′-β-D-2′-deoxyribofuranosyl)-imidazo[1,2-a]-1,3,5-triazin-4 (8H)-one (dP).

As used herein, the term “primer” refers to a short single stranded nucleic acid, or oligodeoxynucleotide (also referred to as an oligonucleotide herein), of about 15 to 30 nucleotides in length, for example. A primer is designed to base pair in a specific or complementary manner to a nucleic acid sequence of interest, and so is considered specific to that nucleic acid. DNA is directional, with the 3′ end of one strand forming base pairs with the 5′-end of the counter strand and a primer is usually designed so that its 5′-end base pairs to the 3′-end of the nucleic acid of interest so that DNA synthesis (which occurs in a 5′ to 3′ direction) to elongate the primer can occur.

As used herein, the terms “oligonucleotide pair”, “oligonucleotide primer pair” and “primer pair” refer to a set of two oligonucleotides that can serve as forward and reverse primers for a nucleic acid of interest. As both strands are copied and amplified in a PCR reaction, each strand requires a primer: the forward primer attaches to the start codon of the template DNA strand (the anti-sense strand), while the reverse primer attaches to the stop codon of the complementary strand of DNA (the sense strand). The 5′-end of each primer binds to the 3′-end of the complementary DNA strand of the nucleic acid of interest.

As used herein, the term “nucleic acid of interest”, or “target”, refers to a polynucleotide sequence, of for example at least one hundred, two hundred, three hundred, four hundred, five hundred or up to one thousand nucleotides in length. The polynucleotide sequence may be specific to a particular organism such as a pathogen, or may be suspected of having a particular mutation along its length, and will encode a particular polypeptide or protein, or mutant form thereof. For example, the polynucleotide sequence may encode the spike protein of SARS-COV-2, or may encode a mutant form of the epidermal growth factor receptor (EGFR) the presence or absence of which renders a patient more or less likely to respond well to cancer treatments such as erlotinib or gefitinib.

As used herein, the terms “capillary pressure barrier”, “capillary valve” or “capillary break”, refer to structural or material modifications used in microfluidics technologies to control fluid flow through a structure, for example a microfluidic channel, or a chamber, and function by increasing the pressure required to further advance the liquid beyond the capillary pressure barrier. This can be achieved by, for example, adjusting the depth or width of the channel or chamber, or by adjusting the contact angle of a liquid with the surface of the channel or chamber. In this way, the liquid is prevented from passing the capillary pressure barrier until an increase in injection pressure is applied to overcome the increase in capillary pressure, or until the contact angle of the liquid with the channel or chamber surface is modified (for example by temperature, as will be described below).

Regardless of end application, PCR subjects a sample to multiple rounds of thermocycling in the presence of an enzyme capable of elongating nucleic acid strands, for example a polymerase. Polymerases catalyse the reaction between a deoxynucleotide triphosphate and a DNA strand, producing an elongated DNA strand bearing one more nucleotide (from the deoxynucleotide triphosphate), and Examples of polymerases used in PCR are pyrophosphate as a by-product. thermostable polymerases such as Taq polymerase (from Thermus aquaticus), Pfu polymerase (from Pyrococcus furiosus), and Bst polymerase (from Bacillus stearothermophilus). The DNA strand that is elongated in PCR is usually in the form of an oligonucleotide primer specific to a target nucleic acid sequence of interest, which is elongated using a mixture of deoxyribonucleotide triphosphates (dNTPs). For full synthesis of a standard DNA strand, four dNTPs corresponding to the four nucleobases found in DNA (adenine, guanosine, thymine and cytosine) are required: 2′-deoxyadenosine 5′-triphosphate, 2′-deoxyguanosine 5′-triphosphate, 2′-deoxycytosine 5′-triphosphate and thymidine 5′-triphosphate.

The three basic steps of a single round or cycle of PCR amplification are denaturation, annealing and chain extension, each optimally taking place at different temperatures (for example in the range of 94-98° C. for denaturation; 50-65° C. for annealing, and 70-80° C. for chain extension, depending on polymerase), hence the term thermocycling. The denaturation step separates the two strands of double-stranded DNA, with each strand acting as a template in the later chain extension step in which a complete complementary strand to the template is produced.

An oligonucleotide primer (comprising for example 15 to 30 nucleotides to ensure a balance of good specificity and efficient hybridization) is annealed to the 3′-end of each single stranded DNA molecule, and acts as a template for the synthesis of the new strand. A DNA polymerase, and a mix of dNTPs then synthesize the new strand in the chain extension step, using the original single strand of DNA as its template. Since both strands of the original DNA duplex are used as templates, a singe round or cycle of PCR results in a doubling of the number of DNA duplexes. The number of copies thus increases exponentially with the number of cycles of amplification: after 2 cycles, four DNA duplexes are present in the sample, while after 3 cycles, 8 duplexes are present.

PCR can be done on a prepared sample of for example 10-50 μL and quantified by monitoring the fluorescence of the fluid as it is thermally cycled. Since the fluorescence is proportional to the amount of nucleic acid (double stranded DNA), the fluorescence intensity increases as the number of cycles of amplification (the amount of double stranded DNA produced) increases. However, in order to achieve a high enough signal to noise ratio, 40 cycles or more are required.

The amplification products (amplicons) are detected optically, for example by using fluorescent reporters. Fluorescent reporter molecules used in PCR include non-specific fluorescent dyes, such as SYBR Green I, which has a distinct emission spectrum when intercalated into any double-stranded DNA, leading to an increase in fluorescence as more double-stranded DNA is produced. Other suitable reporter molecules include target-specific fluorescent reporter molecules, such as the TaqMan hydrolysis probes of target-specific nucleic acids labelled with fluorescent reporter and quencher, with the probe being hydrolyzed by the exonuclease activity of the Taq polymerase, releasing the reporter from the quencher and again leading to an increase in fluorescence.

Reporter molecules may also be linked to a primer to be used in the PCR amplification, such as in the Scorpion® system, a single-stranded bi-labeled fluorescent probe sequence forming a hairpin-loop conformation with a 5′ end reporter and an internal quencher directly linked to the 5′ end of a primer via a blocker (which prevents the polymerase from extending the primer). In the beginning, the polymerase extends the primer and synthesizes the complementary strand of the specific target sequence. During the next cycle, the hairpin-loop unfolds and the loop-region of the probe hybridizes intramolecularly to the newly synthesized target sequence. Now that the reporter is no longer in close proximity to the quencher, fluorescence emission may take place. The fluorescent signal is detected and is directly proportional to the amount of amplified nucleic acid.

Since different reporter dyes have different, and distinct emission spectra, combinations of different reporters can be strategically used in multiplex reactions. In addition to SYBR Green I, other cyanine dyes such as Cy3, or Cy5 can be used, as well as rhodamine dyes. Cy3 has a fluorescence emission at 570 nm, while Cy5 has a fluorescence emission at 670 nm. Other reporter dyes include the Alexa Fluor series of dyes, which have emission wavelengths ranging from 440 nm to 805 nm.

Multiplex PCR is a technique used for amplification of multiple, different, nucleic acid sequences of interest in a single experiment. For example, multiplex PCR may be used to screen for the presence of nucleic acid sequences of interest from multiple, different pathogens in a single reaction, such as simultaneously screening a single sample for the presence of viral nucleic acid sequences from any of SARS-COV, MERS, SARS-COV-2, influenza, and Ebola viruses.

In a multiplex PCR, many different primer pairs are required, with each pair specific to a nucleic acid sequence of interest. For example, if a sample of nucleic acid was being investigated for the presence of 10 different specific nucleic acid sequences of interest (for example 10 different viruses, or 10 different genetic mutations in a patient), then at least 10 different primer pairs would be required for the multiplex PCR.

There is a drive toward the development of new PCR systems, which could be used at the point of care/field of use. Such systems would need rapid thermocycling, in order to amplify the nucleic acid sequences of interest and quickly provide a positive/negative test result.

The present inventors have sought to address these challenges by providing a PCR system with rapid thermocycling capabilities. The proposed system allows for rapid heating via a heater embedded within a microfluidic cartridge and rapid cooling by providing a cooling module.

In one example there is provided a PCR system, comprising:
a microfluidic cartridge having a thermocycling chamber, wherein a floor of the thermocycling chamber is substantially planar and is provided with a heater;
a cooling module configured to engage with and be in thermal contact with a surface of the microfluidic cartridge; and
an optical sensor configured to obtain optical signals from the thermocycling chamber.

In a further example there is provided a method of performing PCR, comprising:
introducing a sample suspected of containing a nucleic acid of interest into a microfluidic cartridge of a PCR system, the PCR system having an optical sensor, and a cooling module in thermal contact with a surface of the microfluidic cartridge, wherein the microfluidic cartridge comprises a thermocycling chamber having a planar floor provided with a heater;
introducing the sample and a PCR Master Mix capable of amplifying the nucleic acid of interest into the thermocycling chamber, to form a reaction mixture in the thermocycling chamber;
subjecting the reaction mixture to thermocycling conditions suitable for amplification by polymerase chain reaction by providing heat from the heater to the reaction mixture and extracting heat from the reaction mixture to the cooling module; and
detecting an optical signal from the reaction mixture.

The system of the present disclosure enables a method having rapid heating rates (˜160° C./s) and rapid cooling rates (30° C./s), allowing for rapid thermocycling and a short time to result (less than 20 minutes). The system also enables sensitive assays to be performed (less than 100 copies/mL of target detected), also exhibiting high specificity.

### PCR System

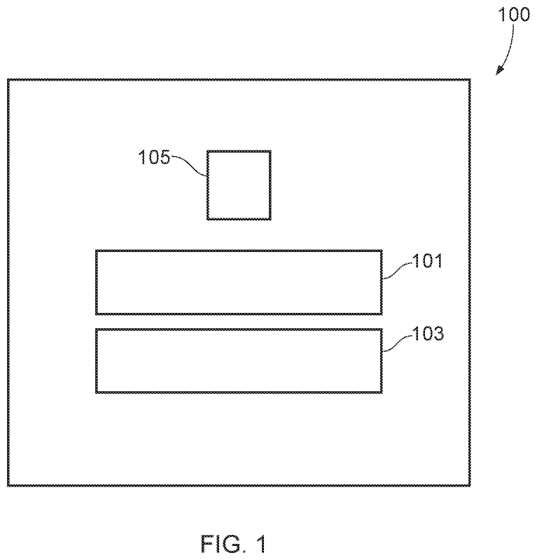

FIG. 1 schematically shows a PCR system 100 in accordance with the present disclosure. PCR system 100 comprises a microfluidic cartridge 101 provided with a thermocycling chamber and a heater (not shown), a cooling module 103, and an optical sensor 105. Although not shown, PCR system 100 may further include electronic circuitry and processing capabilities, as well as fluid driving means and fluid connectors. PCR amplification occurs in the thermocycling chamber, with heating provided by the heater, and heat extraction enabled by cooling module 103.

### Microfluidic Cartridge

Microfluidic cartridge may comprise a single thermocycling chamber or a plurality of thermocycling chambers provided on a substrate. A plurality of thermocycling chambers may be independently operable. In other words, each thermocycling chamber of a plurality of thermocycling chambers may have its own dedicated heater or heaters, so as to be independently controllable. In this way, synchronous or asynchronous control of a plurality of different PCR assays can be performed. Asynchronous control of individual thermocycling chambers within a plurality of thermocycling chambers enables the amplification of a plurality of different samples using different thermocycling, or even isothermal, protocols. In some examples, each thermocycling chamber is provided with a plurality of independently operable heaters or heating elements, thus enabling a plurality of different PCR assays to be performed in a single thermocycling chamber, as described in the methods of the present disclosure. The microfluidic cartridge may be a disposable, or single-use cartridge.

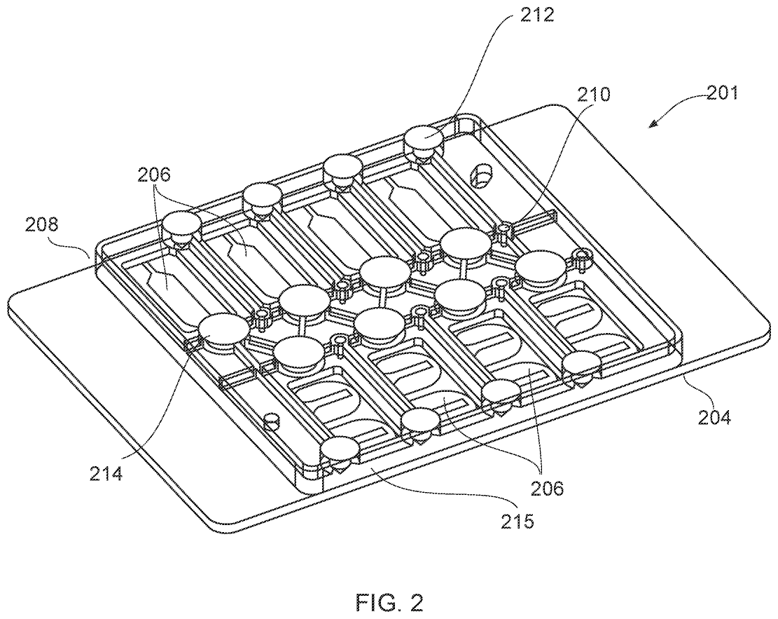

FIG. 2 shows a perspective view of a microfluidic cartridge 201 in accordance with the disclosure. While FIG. 2 shows one example of a microfluidic cartridge, other configurations are possible. For example, microfluidic cartridge 201 comprises a substrate 204 on which a thermocycling chamber 206 is provided. In the example of FIG. 2, eight thermocycling chambers are provided: four elongate chambers in the top row, and four serpentine thermocycling chambers in the bottom row. A floor of thermocycling chamber 206 is substantially planar to ensure an efficient and uniform thermal transfer between a heater and a fluid contained in thermocycling chamber 206. The planar floor ensures that a liquid provided in thermocycling chamber 206 resides as a thin film, maximising rates of thermal transfer during heating and cooling.

Thermocycling chamber 206 forms part of a microfluidic layer 208 of microfluidic cartridge 201. Each thermocycling chamber 206 is provided with a dedicated fluid inlet or port 210, a dedicated fluid outlet or port 212 and a dedicated overflow chamber 214.

In some examples, substrate 204 may be formed from any material suitable for microfluidics, such as glass, silicon, SU-8 (an epoxy-based photoresist material), or polycarbonate. A heater is provided on or within the substrate, to provide heat to the thermocycling chamber. In some examples, the substrate comprises or is a printed circuit board (PCB), and so in some examples is termed a PCB substrate. In some examples, the heater comprises one or more printed electrical traces on a substrate to provide heat to the reaction chamber, or one or more electrical traces etched from or into a conductive material. In some examples, the heater is provided above or below the plane of the microfluidic cartridge. In some examples, the heater is embedded into a substrate on which the thermocycling chamber is disposed. The heater may be embedded into the substrate by machining or etching portions of the substrate into which the heater can be embedded, or by encapsulating the heater in a liquid precursor material that can be solidified or cured to form the substrate. A suitable material is SU-8, which is a liquid, until cured by UV light. In other examples, the heater is provided on a surface of the substrate. In some examples, the heater comprises a flat panel heater or one or more thermally conductive printed or etched electrical traces. In some examples, the heater comprises a Peltier device, a flat panel heater in the form of a solid-state active heat pump. The heater may be formed of any thermally conductive material, such as copper, or gold or silver.

The heater may be in the form of a plurality of thermally conductive printed or etched electrical traces. The plurality of thermally conductive traces may be arranged parallel to each other in a single plane, and be spaced from each other in a horizontal direction by at least 5 μm, for example at least 10 μm, for example at least 50 μm, for example at least 100 μm, for example at least 200 μm. The plurality of thermally conductive traces may be spaced from each other by a distance in the range of from 5 μm to 200 μm, for example 10 μm to 100 μm.

In other examples, the plurality of thermally conductive traces may be arranged in different or multiple planes, and be termed a bi-layer or multi-layer heater. The traces of a bi-layer or multi-layer heater may be spaced apart in the vertical direction by at least 5 μm, for example at least 10 μm, for example at least 50 μm, for example at least 100 μm, for example at least 200 μm. Each layer may be spaced from another layer in the vertical direction by a distance in the range of from 5 μm to 200 μm, for example 10 μm to 100 μm.

In some examples the heater comprises at least partially overlapping conductive traces provided in different layers of the microfluidic cartridge. For example, the bi-layer or multi-layer heater may comprise a plurality of thermally conductive traces in which the traces of one layer run parallel to and at least partially or completely overlie the traces of a second layer.

Alternatively, the bi-layer or multi-layer heater may comprise a plurality of thermally conductive traces in which the traces of one layer run perpendicular or substantially perpendicular to the traces of a second layer. In other examples, the bi-layer or multi-layer heater may comprise a plurality of thermally conductive traces in which the traces of one layer run parallel to the traces of a second layer and partially overlap or do not overlap with the traces of the underlying layer. In this example, viewed from above or below the two planes, the two sets of traces may resemble interdigitated electrical traces. The heater may comprise a bi-layer or multi-layer configuration of electrical traces in which a series of printed or electrical traces are overlaid with a diffuser layer or heat spreader of thermally conductive material which acts to diffuse or spread heat from the traces to a fluid in the overlying thermocycling chamber. Thus, the heater can comprise a series of electrical traces in a first layer of the microfluidic cartridge overlaid with a diffuser of thermally conductive material in a second layer of the microfluidic cartridge.

The diffuser layer may be a passive heat spreader, and be formed from the same thermally conductive material as the thermally conductive material of the printed traces, for example deposited copper which can be etched into any required pattern. In some examples, one or more heater elements comprises a resistive heater. Etched copper traces are examples of resistive heaters, as they not only dissipate heat, but their resistance changes with temperature.

Resistive heaters of this type are particularly suited for use in the thermocycling chamber of the present disclosure as they can be used not only as heaters, but simultaneously also be used as temperature sensors to monitor temperature within thermocycling chamber 206 and allow immediate and accurate feedback control. Since copper does not affect a PCR reaction, it is also suited to be a heater in direct contact with a fluid within thermocycling chamber 206, thereby further improving thermal transfer and heating rates. The heater may be in the form of one or more serpentine traces. It will be understood that the term “serpentine” refers to a single trace having a plurality of parallel sections joined at their ends to neighbouring sections. The heater may comprise more than one serpentine trace, in different planes of substrate 204. In one configuration, an outer or upper serpentine trace may be provided on an upper surface of substrate 204, which may be a PCB substrate, so as to be in direct contact with a fluid of thermocycling chamber 206, with an inner or lower serpentine trace embedded into, for example, a dielectric layer of substrate 204. Thus, the heater may comprise one, two, or more than two, for example at least two serpentine traces. The at least two serpentine traces may be oriented perpendicular to each other and be provided in different layers or planes of substrate 204. The heater may comprise at least two perpendicularly oriented serpentine traces provided in different layers or planes of the microfluidic cartridge.

In some examples, the footprint of the heater extends beyond the footprint of thermocycling chamber 206, to ensure that any thermal edge effects are avoided and to ensure uniform heating in thermocycling chamber 206. As used herein, the term “footprint” refers to the area of the microfluidic device covered by, taken up or occupied by the component in question, corresponding to the physical layout of the component, for example in the X-Y plane or horizontal dimension. Thus, the “footprint” of the heater corresponds to the maximum dimensions of the heater, for example provided for by the maximum length and the maximum width of a serpentine trace. The footprint of the thermocycling chamber may therefore correspond to the area provided by the internal length and width of the thermocycling chamber. The heater footprint may be at least 5% greater than the footprint of thermocycling chamber 206, for example at least 10% greater, for example at least 15% greater, for example at least 20% greater, for example at least 25% greater, for example at least 30% greater, for example at least 40% greater, for example at least 50% greater. The heater may have an area, or a footprint, of from 50 mm² to 600 mm², for example from 60 mm² to 550 mm², for example from 70 mm² to 500 mm², for example from 75 mm² to 475 mm², for example from 80 mm² to 460 mm². The heater footprint dimension may be determined by the size of the thermocycling chamber to be heated, and may in some instances be dimensioned so as to have an overall footprint of 84 mm² (such as a 6×14 mm heater), or to have an overall footprint of 450 mm² (such as a 15×30 mm heater).

Regardless of configuration, the heater may receive electrical power from electrically conductive wires provided on or to the microfluidic cartridge to form an electrical circuit which supplies electrical current to the heater. Such components may be controlled by a controller located on or off the microfluidic cartridge via control signals. Heater configurations as described above enable temperature ramp rates of from 20 to 200° C. per second.

A dielectric layer may be disposed over the substrate and/or the heater embedded in or on the substrate. The dielectric layer may be spun on or sputtered onto the substrate. Thus, in some examples, the substrate comprises a dielectric coating of polyimide, SU-8, silicon oxide, silicon nitride, aluminium oxide, aluminium nitride or any combination/stack thereof. Another suitable material is Kapton®, which may be incorporated into a coating or stack with any of the aforementioned materials.

Thermocycling chamber 206 can be provided in a microfluidic layer 208 (also termed a microfluidic stack) of microfluidic cartridge 201, disposed on substrate 204. As used herein, the terms “microfluidic layer”, “microfluidic stack”, “fluidic layer” or “fluidic stack” refer to the components of the microfluidic cartridge through which one or more fluids can pass during use of the microfluidic cartridge, for example through one or more microfluidic channels and chambers. The terms are intended to encompass multiple flow paths, for example in different levels of the layer/stack, and distinguish these flow channel-containing components from other operational modules such as electronic circuitry and sensors. The microfluidic layer or microfluidic stack may comprise any material or combination of materials suitable for use in microfluidic cartridges, including polycarbonate, and cyclic olefin copolymer (COC). In some examples, thermocycling chamber 106 is formed in a microfluidic layer or microfluidic stack by moulding, or selectively etching or machining away regions of material so as to form a thermocycling chamber. In some examples, thermocycling chamber 206 is formed wholly within the material forming the microfluidic layer (for example COC), with that material also forming the base of thermocycling chamber 206. In other examples, the material forming the microfluidic layer forms the walls of thermocycling chamber 206, with the underlying substrate (for example with a dielectric layer as described) forming the base or floor of thermocycling chamber 206. In other examples, thermocycling chamber may be provided in one or more layers of pressure-sensitive adhesive that bonds the upper layers of the microfluidic stack to substrate 204, with the upper surface of substrate 204 forming the base or floor of thermocycling chamber 206. Other layers present in a microfluidic stack may include layers of adhesive to bond the microfluidic layer to the substrate and/or bond layers of a microfluidic stack to each other. Suitable adhesives include pressure-sensitive adhesives, which can comprise an elastomer based on acrylic, silicone or rubber optionally compounded with a tackifier such as a rosin ester. Convenient pressure sensitive adhesives are in the form of double-sided films or tape, such as the acrylic adhesives 200MP and 7956MP available from 3M™. Microfluidic stack 208 may include a transparent lid. The lid may be transparent in the visible region of the electromagnetic spectrum, and have low levels of autofluorescence to allow detection of fluorescence in thermocycling chamber 206. The lid may be formed from any suitable material, for example a thermoplastic material such as polycarbonate or

COC as described above.

Thermocycling chamber 206 may have an internal volume of less than 100 μL, for example less than 75 μL, for example less than 50 μL, for example less than 30 μL, for example less than 20 μL, for example about 15 μL, depending on configuration. For example, the serpentine thermocycling chamber in the cartridge of FIG. 2 have an approximate volume of 30 μL, while the elongate chambers have an approximate volume of 15 μL.

Thermocycling chamber 206 may also be referred to as a flow cell, and is may be dimensioned so as to have an aspect ratio of its largest dimension, for example in the XY plane, to its height of from 10:1 to 1000:1, for example from 50:1 to 1000:1, for example from 100:1 to 1000:1, for example from 500:1 to 1000:1. In some examples, the aspect ratio may be from 10:1 to 900:1, for example from 10:1 to 750:1, for example from 10:1 to 500:1, for example from 10:1 to 250:1, for example from 10:1 to 150:1. Thinner fluid thicknesses allow for a more rapid, and more uniform heat transfer from an embedded heater in microfluidic cartridge 201, and a subsequent more rapid, and more uniform heat transfer from the fluid to a cooling module. In some examples, thermocycling chamber 206 has a height of less than 500 μm, for example less than 400 μm, for example less than 300 μm, for example less than 200 μm, for example a height of from 500 μm to 50 μm, for example a height of from 400 μm to 75 μm, for example a height of from 300 μm to 100 μm, for example a height of from 200 μm to 100 μm, for example a height of about 150 μm to 200 μm.

In some examples, microfluidic layer 208 is provided with one or more fluid inlets 210 and outlets 212 to provide a liquid such as a reaction liquid to the or each thermocycling chamber. In some examples, the outlet may also be referred to as a vent. The presence of a vent enables unhindered flow of liquid through the thermocycling chamber and minimises risk of unwanted bubble formation within the thermocycling chamber. The outlet or vent may be provided with a membrane. The membrane may be a gas-permeable membrane, a flexible membrane, a rupturable or non-rupturable membrane or a membrane which exhibits one or more of these properties. The membrane may be formed of a hydrophobic material, or a hydrophilic material.

In some examples, the or each thermocycling chamber is also provided with an overflow chamber 214, which may be a dedicated overflow chamber or an overflow chamber common to all thermocycling chambers. The overflow chamber may be provided in a different layer of the microfluidic stack. Depending on use, one or both of outlet 212 and overflow chamber 214 may be provided with a permeable or non-permeable, or rupturable or non-rupturable seal.

Thermocycling chamber 206 may be provided with at least one capillary break on an internal surface of the thermocycling chamber. The at least one capillary break controls the location of the liquid-gas interface during filling to avoid bubble formation.

In some examples, the thermocycling chamber has a high aspect ratio of at least 1:10 (height to width) and is provided with at least one capillary break on an internal surface of the thermocycling chamber, to control the location of the liquid-gas interface during filling to avoid bubble formation. In some examples, the at least one capillary break comprises a region of raised material on, or depressed material in, the floor or ceiling of the chamber. In some examples, the at least one capillary break can be a region of the floor or ceiling of the thermocycling chamber with a different contact angle to the contact angle of the surrounding floor or ceiling, for example by depositing or printing a material having a higher hydrophobicity than the surrounding material. For example, the at least one capillary break may be formed by depositing at pre-defined locations a hydrophobic material onto a dielectric layer. The dielectric layer and the hydrophobic material may be as described. In some examples, the at least one capillary break comprises a resistor.

In some examples, the thermocycling chamber is provided with one or more thermally controllable capillary breaks on an internal surface thereof. In some examples, the at least one capillary break is thermally activated or controllable, thereby providing control over when an infilling liquid is able to pass or burst the at least one capillary pressure barrier. In some examples, the at least one capillary break is thermally activated via optical means, for example via selective absorption of radiation from an optical source. For example, the at least one capillary break may be formed of a material that selectively absorbs light of a particular wavelength and thereby generates heat. Selective absorber materials include ceramic, or metal oxide, materials such as copper oxide or cobalt oxide deposited on a substrate. In some examples, when the at least one capillary break comprises an electrode assembly (for example a plurality of printed electrical traces) the at least one capillary break is thermally activated by providing an electrical current to the electrode assembly. In some examples, the at least one capillary break comprises an electrode assembly, for example a printed electrode on a PCB substrate. In particular configurations of microfluidic cartridge, an overflow chamber may be associated with a thermocycling chamber, with a capillary break located at the inlet to the overflow chamber and configured to control flow of incoming liquid into the thermocycling chamber and only permitting liquid to enter the overflow chamber once the thermocycling chamber is filled.

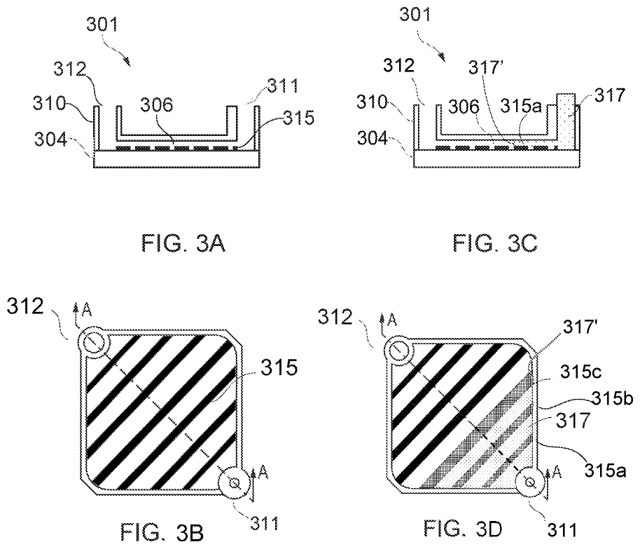

FIGS. 3A and 3B show a thermocycling chamber 306 of a microfluidic cartridge 301, with FIG. 3A showing a cross-section slice of the plan view of FIG. 3B along the dotted line A-A and FIG. 3C showing a cross-section slice of the plan view of FIG. 3D along the dotted line A-A. FIGS. 3C and 3D show thermocycling chamber 306 of FIGS. 3A and 3B respectively during filling of the thermocycling chamber. Thermocycling chamber 306 is of a different configuration to that of thermocycling chamber 206 of FIG. 2, though the operation and function of these different configurations is within the present disclosure. In FIGS. 3A and 3B, substrate 304 is provided with a plurality of capillary breaks 315 on an inner surface of the thermocycling chamber, which is formed in a fluidic stack 310. Thermocycling chamber 306 is also provided with fluid inlet 311, and an outlet 312, which may also be termed an exhaust or vent. In the system of FIGS. 3A to 3D, inlet 311 and outlet 312 are dedicated ports from above for thermocycling chamber 306, though it will be appreciated that in other configurations, a fluid inlet may for example be in the same plane as the thermocycling chamber.

In FIGS. 3A to 3D, the at least one capillary break comprises an electrode assembly in the form of printed electrical traces on the PCB substrate, which have a raised profile above the surface of the substrate. The electrode assembly functions as the at least one capillary break to control filling of the liquid volume into thermocycling chamber 306. In some examples, the electrode assembly comprises one or more electrodes that extend across the entire width of thermocycling chamber 306 perpendicular to the direction of filling (that is, perpendicular to flow axis from an inlet to an outlet). In some examples, the electrode assembly comprises a plurality of connected printed electrical traces, which function as the at least one capillary break. In some examples, the electrode assembly comprises a plurality of independently operable printed electrical traces, each of which functions as a capillary break. In some examples, each printed electrical trace of the independently operable printed electrical traces extends across the entire width of thermocycling chamber 306 perpendicular or normal to the direction of filling. The electrical traces which form capillary breaks 315 are connected to electrical contacts and a power source to complete the electrical circuits, though for convenience these are not shown in FIGS. 3A to 3D.

In operation, the liquid volume enters the thermocycling chamber and the liquid-gas interface is pinned at a first capillary break (for example the first electrical trace). A short thermal pulse quickly raises the temperature locally to the capillary break at this interface (but negligibly elsewhere), locally reducing the surface tension, and so allowing the interface to unpin. The unpinning may initially occur at a particular section of the capillary break and then propagate along the length of the capillary break. The preferential unpinning at a location may be controlled by a lower difference in surface tension or a lower step height of the capillary break. Such a mode of unpinning may be used to control how the liquid fills the region between sequential capillary breaks.

The liquid volume then proceeds by capillary action to further fill the chamber, until the next trace. The next pulse then unpins the interface there and the process repeats. The pulses are timed to allow the fluid to fill the region between the traces.

In the plan views of FIGS. 3B and 3D, it can be seen that the plurality of capillary breaks 315 are oriented perpendicular to a fluid flow, with infilling liquid 317 entering via inlet 311. In the side view of FIG. 3C, and the plan view of FIG. 3D, the liquid front 317′ of liquid 317 can be seen pinned against the downstream edge of capillary break 315c, having advanced past capillary breaks 315a and 315b that have already been thermally actuated to let liquid front 317′ pass. Capillary breaks 315a, 315b and 315c are shown with hatching in FIG. 3D as these are all upstream of liquid front 317′ (i.e. close to inlet 311) and so are covered with liquid 317, as can be seen in FIG. 3C. Upon thermal actuation of capillary break 315c, liquid front 317′ will advance further through thermocycling chamber 306 until it reaches the next capillary break.

In some examples, the thermocycling chamber is provided with a plurality of capillary breaks on the floor of the thermocycling chamber. In some examples, the thermocycling chamber is provided with a plurality of capillary breaks on the ceiling of the thermocycling chamber. In some examples, the thermocycling chamber is provided with a plurality of capillary breaks on the floor and the ceiling of the thermocycling chamber. In some examples, the plurality of capillary breaks on the floor and the ceiling of the thermocycling chamber are aligned opposite to each other, or in an alternating pattern. In some examples, the plurality of capillary breaks are independently or commonly activated by electrical heating, or by optical heating as described herein.

In some examples, the electrode assembly performs multiple functions, including thermal control of the filling of the thermocycling chamber, and thermal control of the liquid volume during a thermocycling reaction.

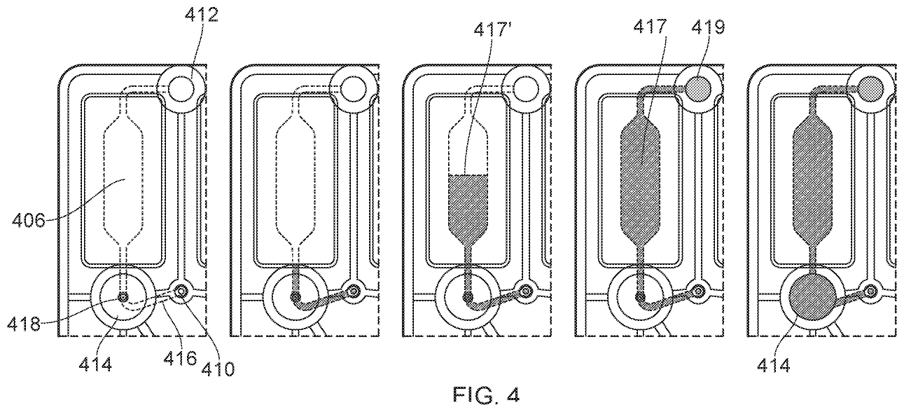

FIG. 4 illustrates the sequence of filling of a thermocycling chamber. In the first image, inlet 410 is shown, with a fluid channel 416 leading toward overflow chamber 414 and onward to thermocycling chamber 406. Fluid channel 416 and thermocycling chamber 406 are provided in an underlying layer of the fluidic stack. Overflow chamber 414 is provided in an upper layer of the fluidic stack, and is connected to fluid channel 416 via an inlet which is constricted (for example by having a narrower diameter or dimension) relative to fluid channel 416. Due to capillary forces, the pinhole inlet serves as a capillary break 418, preventing fluid flow into overflow chamber 414 and instead directing flow of infilling liquid 417 from inlet 410 into thermocycling chamber 406 until thermocycling chamber 406 is full, as is shown in the second and third images of FIG. 4. The fourth image shows that continued fluid infill causes the front to reach outlet 412. In the configuration of FIG. 4, outlet 412 is provided with a membrane or sealing plug 419 of flexible but non-rupturable material, for example a polyethylene or a PTFE (e.g. Goretex®) plug which expands as fluid infill continues, as shown in the fourth image, leading to an increase in flow resistance. The increase in flow resistance continues until the back pressure is high enough for the fluid to breach capillary break 418 and overflow into overflow chamber 414. As can be seen in FIG. 2, each thermocycling chamber 206 is provided with an overflow chamber 214, with flow channels from each overflow chamber connecting the individual overflow chambers to a common reservoir 215 extending around the perimeter of the fluidic stack or microfluidic layer 208. In other configurations, for example in which overflow chamber 414 is not present, sealing plug 419 need not be included, with infilling liquid flowing through outlet 412 once thermocycling chamber 406 has filled.

Although not shown in FIG. 4, thermocycling chamber 406 may be provided with a series of capillary breaks as described above, each extending across the width of the chamber, providing a plurality of locations at which the infilling liquid front 417′ is temporarily halted until the front extends across the length of the capillary pressure break.

The microfluidic cartridge may be in the form of a cassette, or chip, to be used in combination with other components of the the PCR system, in particular the cooling module and optical sensor. In some examples, the microfluidic cartridge may be a single use or disposable cartridge. In some examples, the microfluidic cartridge may be configured to be inserted into or received by a port in system. In some examples, the microfluidic cartridge may be provided with one or more fluidic connections that are configured to engage with one or more corresponding fluidic connections in system, to enable fluid flow from the system into the microfluidic cartridge, for example to enable transfer of a sample injected into an injection port of the system to be transferred to a thermocycling chamber of the microfluidic cartridge. In other examples, the or each thermocycling chamber of the microfluidic cartridge may be filled with sample prior to inserting the microfluidic cartridge into the system, for example by manual pipetting a sample solution through an inlet port such as a Luer connector or membrane valve.

In some examples a reaction reagent, for example one or more components of the PCR Master Mix, is provided in lyophilised form on an internal surface of the thermocycling chamber. In some examples a reaction reagent, such as a component of the PCR Master Mix is disposed on at least one inner surface of a reagent storage chamber or reservoir provided on the microfluidic cartridge and which chamber or reservoir is fluidly connected to a thermocycling chamber. In some examples, the reaction reagent may be disposed on the base or floor of the thermocycling chamber. The reaction reagent may be covalently or non-covalently attached to the base or floor of the thermocycling chamber. In the instance that the reaction reagent is non-covalently attached, the process of lyophilising a solution of the reaction reagent present on teh base or floor of the thermocycling chamber results in deposition of the solid reaction reagent. Covalent attachment of the reaction reagent can be achieved, for example, through the use of a thiol-modified surface and a thiol-modified reaction reagent, resulting in covalent attachment via a disulfide bond, or a gold surface wtih a thiol-modified reaction reagent. In some examples, the at least one reaction reagent may also be positioned on other inner surfaces, such as peripheral walls of the thermocycling chamber provided by a microfluidic layer extending from the substrate to the lid as described herien. In some examples, the at least one inner surface of the thermocycling chamber comprises multiple reaction reagents wherein each of the reaction reagents is disposed at a different location on the at least one inner surface of the thermocycling chamber. In some examples, each reaction reagent is a different reaction reagent. By providing different reaction reagents at different locations, a single-chamber multiplexed reaction is enabled.

The reaction reagent may comprise a chemical or biological material that is to be used in a chemical or biological reaction to take place in the thermocycling chamber. In some examples, the reaction reagent is introduced into the thermocycling chamber as a fluid and subsequently freeze-dried to form a freeze-dried, i.e. lyophilized, reaction reagent on a designated portion of the interior surface of thermocycling chamber 106. For example, a solution of reaction reagent in a suitable solvent can be pipetted onto the interior surface of the thermocycling chamber during production of the microfluidic cartridge, with the solvent subsequently being evaporated off (for example by freeze drying) to leave the deposit of reaction reagent in solid form. Thus, in some examples, the reaction reagent comprises a freeze-dried or lyophilized reaction reagent.

In some examples, the reaction reagent may be a nucleic acid, for example a single strand of DNA or RNA. In some examples, the at least one reaction reagent is a single stranded oligonucleotide, for example an oligo (deoxy) nucleotide, that can be used as a primer in a PCR reaction. The oligonucleotide may be a first primer of a primer pair for a PCR reaction, when the second primer of the primer pair is introduced into the thermocycling chamber separately. Thus, In some examples, the reaction reagent is dissolved in a suitable aqueous or organic solvent in order for it to be conveniently deposited, for example water, or an aqueous buffer solution such as TE buffer (Tris-EDTA), TAE buffer (Tris-acetic acid-EDTA) or TBE buffer (Tris-borate-EDTA), and deposited by manual or robotic pipetting onto a surface of a substrate or other surface which will form the surface of the thermocycling chamber. The component of the microfluidic cartridge bearing the surface with deposited reaction reagent is subsequently subjected to freeze-drying (lyophilization). In the example in which the reaction reagent is a single strand of nucleic acid, the use of such buffers can stabilize the lyophilized nucleic acid. As described above, lyophilization results in a non-covalent attachment of the nucleic acid to the surface, though covalent attachment through, for example, disulfide linkages between a thiol-modified surface and thiol-modified nucleic acid can result in a covalent attachment. The resulting disulfide attachment of the nucleic acid can be reversed under reducing conditions, thus releasing the nucleic acid into solution, if required.

In some examples, the inner surface comprises a plurality of positions (or spots) of disposed reaction reagent as described herein, with each spot being spatially separated from the other spots. The multiple reaction reagents or spots may be disposed at a location of from 100 to 500 μm apart from each other, for example from 200 to 500 μm apart, for example from 300 to 500 μm apart. By spacing the reagents apart and having no fluid flow during a reaction, cross-contamination between multiple reactions is avoided. The spacing between individual spots of deposited reaction reagent will depend on the molecular size or weight of the particular reaction reagent. The larger the molecule, the less distance it will travel by simple diffusion, and the less spacing is required between adjacent spots of reaction reagent. For example, an oligonucleotide that may serve as a primer in a PCR reaction will in the absence of any forced fluid flow diffuse through a liquid at most 100 μm for a fast PCR reaction taking 10 minutes.

An individual film of thermally dissolvable or degradable film can be applied over the individual spots of reaction reagent. The thermally dissolvable or degradable film may cover the reaction reagent. The film is provided in an amount sufficient to isolate the reaction reagent between the film and the surface of the thermocycling chamber. Further details on the thermally dissolvable or degradable film will be described later.

In some examples, a plurality of reaction reagents are disposed on the at least one inner surface at discrete, spaced apart locations, wherein each reaction reagent is a different reaction reagent. In some examples, each location comprises a different reaction reagent. As different locations in the thermocycling chamber may comprise different reaction reagents, each location can be used for a different, specific reaction. For example, in the situation in which each reaction reagent is an oligodeoxynucleotide that can be used as a PCR primer in a PCR reaction, with different oligodeoxynucleotides being specific to different nucleic acid sequences of interest, a multiplexed PCR reaction for investigating the presence of different nucleic acid sequences of interest in a single sample can occur in the thermocycling chamber. As noted above, spacing of the different reaction reagents based on their limits of diffusion avoids cross-contamination of reactions.

In other examples of microfluidic cartridges as described herein, barriers or flow structures are also provided between certain reaction reagent locations, to further restrict any cross-reactivity or cross-contamination between reagent locations. The barriers may be formed on the surface of the thermocycling chamber prior to deposition of any reaction reagent. In some examples, the barriers are integrally formed as part of the substrate on which the thermocycling chamber is disposed by moulding or etching (for example by laser micromachining) the substrate. In some examples, the barriers are deposited or affixed to the substrate in a separate manufacturing step. The barriers may be formed from the same material as the substrate, or walls of the reaction chamber, or from any other suitable material such as deposited or 3D printed metal, ceramic or polymeric resin.

The barriers may be formed with a parallel flow design, or with a serpentine flow design. In some examples, the locations of reaction reagent may be provided with their own individually addressable heater. In this way, individualised heating protocols can be established for each location, enabling for example different PCR thermocycling protocols to be followed, or for different thermally dissolvable or degradable films which require different temperatures for dissolving and/or degrading to be used at different locations.

In some examples, a thermally dissolvable or degradable film can be applied to a larger area of the at least one inner surface of the thermocycling chamber, so as to cover or isolate all reaction reagent locations under one continuous film. As used herein, references to the at least one reagent being isolated by the thermally dissolvable or degradable film are to the reagent being in direct contact with the at least one inner surface of the thermocycling chamber and the thermally dissolvable or degradable film, rather than being fully encapsulated by the thermally dissolvable or degradable film in a free moving particle.

As used herein, a thermally dissolvable film is a material which is insoluble in a solution of reactants or reagents introduced into the thermocycling chamber until the solution is thermally actuated. In other words, a thermally dissolvable film may dissolve when in contact with a reaction solvent, for example water, and when the substrate upon which it is positioned is heated so that the temperature of the reaction solution or solvent increases.

As used herein, a thermally degradable film is a material which is stable in a solution of reactants or reagents introduced into the thermocycling chamber until the solution is thermally actuated. In other words, a thermally degradable film may degrade when in contact with a reaction solvent, for example water, and when the temperature of the reaction solution or solvent increases. In some examples, a thermally degradable film is degraded by the action of one or more degrading enzymes as will be described later. For example, a degrading enzyme may be disposed on the surface of the thermocycling chamber with the reaction reagent and isolated by the thermally degradable film.

The action of heating up the film when in contact with the reaction solvent softens the film to an extent that the degrading enzyme is no longer isolated and is redissolved in the reaction solvent and can thereby begin degrading the film. In some examples, the thermally dissolvable film is also a thermally degradable film in the presence of one or more degrading enzymes. The thermally dissolvable film may be degraded using one or more degrading enzymes after dissolving of the film. Degrading of the film prior to any thermocycling ensures that the dissolved polymer cannot indiscriminately bind to any nucleic acid strands and inhibit amplification.

In some examples, the temperature to which the reaction or reaction mixture is heated may depend upon the composition of the film. In some examples, the reaction or reaction mixture is heated to a temperature of from 40 to 120° C., for example from 50 to 110° C., for example from 60 to 100° C., for example from 70 to 90° C. In some examples, the dissolution temperature may be from 90° C. to 100° C. The thermally dissolvable or degradable film protects the at least one reaction reagent. In some examples, the thermally dissolvable or degradable film protects nucleic acid strands such as PCR primers, or multiple different PCR primers, from premature dissolution by an aqueous sample solution washing over the region when filling the chamber.

In some examples, the thermally dissolvable or degradable film comprises polyvinyl alcohol, polyvinyl acetate, cellulose, polyester, polyethylene terephthalate, polyurethane or combinations thereof. In some examples, the thermally dissolvable or degradable film comprises polyvinyl alcohol. In some examples, the polyvinyl alcohol comprises acetyl groups. In some examples, the polyvinyl alcohol does not comprise acetyl groups. In some examples, the polyvinyl alcohol is cross-linked. In some examples, the polyvinyl alcohol is not cross-linked. In some examples, the polyvinyl alcohol is Vinex® 1003 sold by Air Products Co, or Elvanol®, a fully hydrolysed polyvinyl alcohol sold by DuPont.

In some examples, the thermally dissolvable or degradable film comprises polyvinyl acetate, for example highly crystallized totally saponified polyvinyl acetate. In some examples, the thermally dissolvable or degradable film comprises cellulose, for example, a cellulose such as nitrocellulose.

In some examples, the thermally dissolvable or degradable film comprises one or more polymers, for example, the polymer may comprise, but is not limited to, polyester, polyethylene terephthalate and polyurethane, or combinations thereof. In some examples, one or more degrading enzymes are used to degrade these types of polymer by cleaving bonds between the monomeric units of the polymer. In some examples the degrading enzymes for degradation of polymer may include, but are not limited to cutinases (for the break-down of polyester through hydrolysis of the ester groups), polyesterases (for hydrolysis of aromatic polyesters such as polyethylene terephthalate), and enzymes incorporating polyhydroxyalkanoate binding modules (such as a polyamidase conjugated to the polyhydroxyalkanoate binding module for polyurethanes).

In some examples, the thermally dissolvable or degradable film comprises polyvinyl alcohol and the degrading enzyme involved with the degradation is polyvinyl alcohol oxidase or polyvinyl alcohol hydrogenase.

In some examples, the action of heat on the reactant solution softens and separates the film from the at least one inner surface to the extent that a degrading enzyme disposed underneath the film is then dissolved into solution and can degrade the film. In these examples, the degrading enzyme may be in solution with the reaction reagent when pipetted onto the surface or may be added as part of a separate solution which is then freeze-dried as described. The degrading enzyme may be deposited on top of, or adjacent to the reaction reagent. In some examples, a degrading enzyme is provided as part of the reaction solution that is introduced into the reaction chamber.

### Cooling Module

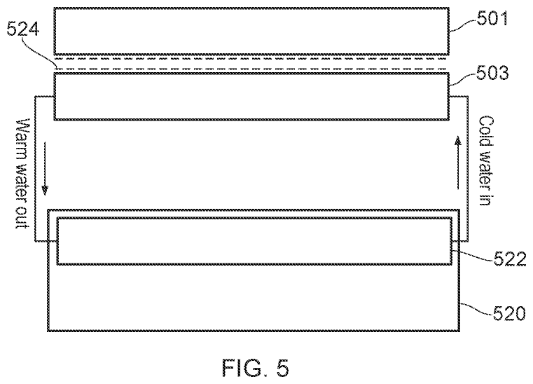

PCR system is provided with a cooling module 103 to enable rapid and efficient cooling of a PCR mixture undergoing amplification in a thermocycling chamber of the microfluidic cartridge. Cooling module 103 may be any suitable cooling module that when in thermal contact with microfluidic cartridge 101 enables good thermal transfer from microfluidic cartridge 101. As has been described above, the heater of the microfluidic device provides heat to the thermocycling chamber in order to reach denaturing conditions of a PCR mixture. In order to rapidly cool the PCR reaction mixture to an annealing temperature, rapid heat transfer away from the PCR mixture through thermal contact between the microfluidic device and the cooling module is required. As used herein, two components (for example a cooling module and a microfluidic cartridge) are in thermal contact with one another if heat transfer occurs when one component is at a higher temperature than the other component. Efficient thermal transfer can be achieved by having cooling module 103 in direct (e.g. physical) contact with microfluidic cartridge 101 for conductive cooling, although cooling modules that cool microfluidic cartridge 101 via convective means are also suited. It will be understood that “direct” contact with microfluidic cartridge 101 does not exclude the presence of intermediate materials so long as these are also thermally conductive and enable, rather than prevent, heat transfer. Efficient thermal transfer can be increased by providing clamping means to clamp the microfluidic cartridge to the cooling module, to maintain good thermal contract. Example cooling modules are depicted in FIGS. 5 to 7.

FIG. 5 shows a cooling module 503 in thermal contact with a microfluidic cartridge 501. Cooling module 503 forms part of a coolant circuit with refrigeration system 520 through which a working fluid is cycled. Refrigeration system 520 includes a radiator 522, which allows warm working fluid (in this example water) received from cooling module 503 to be refrigerated before being returned to cooling module 503. Water is only one example of a working fluid or coolant, with other refrigerants including glycols (e.g. ethylene glycol or propylene glycol), which may be used alone or blended with water. In FIG. 5, a layer of compliant thermal interface material 524 between cooling module 503 and microfluidic cartridge 501 is shown, though this layer is optional. If included, compliant thermal interface material may be any thermally conductive material that enables good thermal contact and transfer, such as indium and indium alloys, such as In—Ag; gallium and gallium alloys; copper, aluminium, and lead. Suitable materials also include greases or polymer suspensions of silver, carbon micro- and nano-particles, aluminium oxide, boron nitride, zinc oxide, aluminium nitride, where the polymer material can be epoxy-based, silicone-based, urethane-based, and/or acrylate-based.

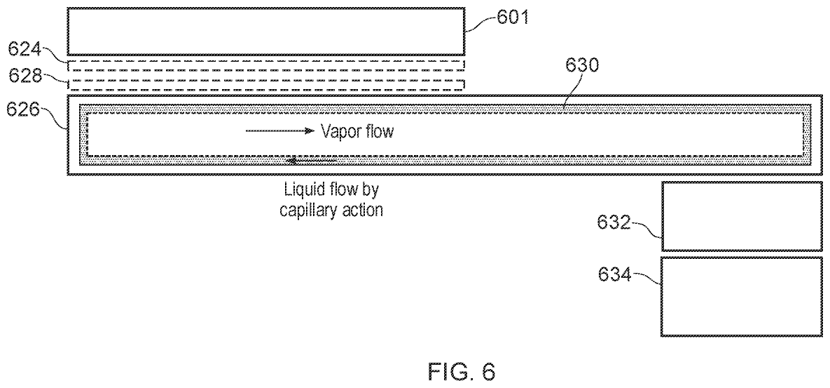

FIG. 6 shows a cooling module in thermal contact with microfluidic cartridge 601. In FIG. 6, the cooling module is in the form of heat pipe 626, in which a working liquid (such as water or another coolant) flows along a porous liquid path on an internal surface of the pipe, for example by capillary flow. Heat is transferred from microfluidic cartridge 601 to heat pipe 626, causing the working fluid to evaporate, with the vapor flowing counter-current to the liquid flow. As the vapor travels to the end of the heat pipe furthest from the region which contacts microfluidic cartridge 601, it condenses on the inner surface of heat pipe 626, aided by heat sink 632 and fan 634, and in the process releasing the latent heat that was absorbed from microfluidic cartridge 601. Heat pipes can be constructed from copper for water-based systems, but can also be constructed from aluminium, steel, or other thermally conductive materials. While water has been mentioned as a working liquid for heat pipe 126, other working liquids which can readily be vaporized and condensed include ammonia.

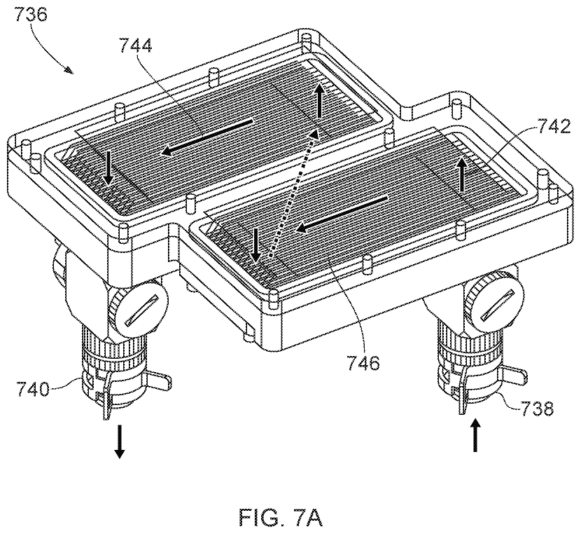

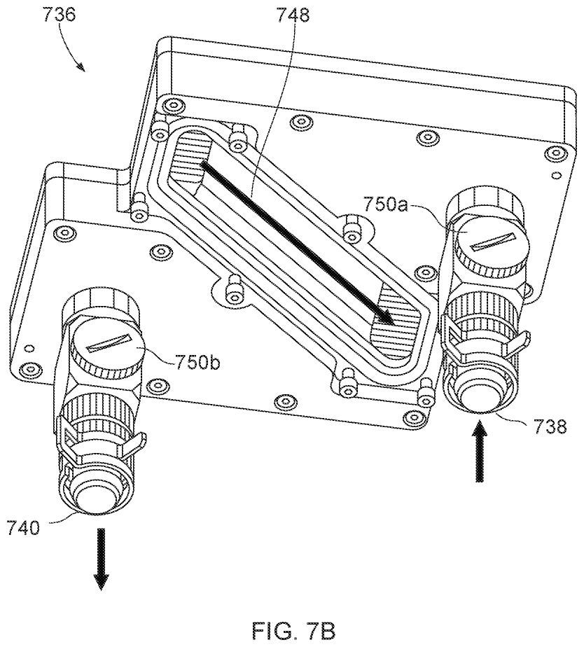

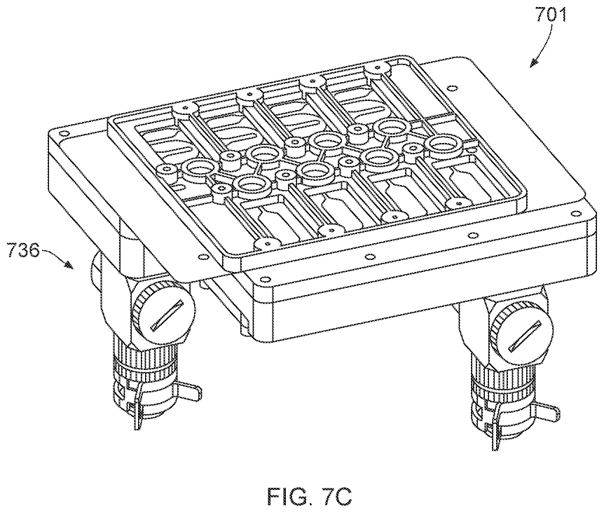

The cooling module may be configured based on the configuration of the microfluidic cartridge to be used. For example, if the microfluidic cartridge comprises a plurality of thermocycling chambers provided in at least two groups, the cooling module may comprise a number of cooling panels equivalent to the number of groups of thermocycling chambers. Having two cooling panels, each serving a group of thermocycling chambers together enables, for example, different heating and cooling protocols to be performed on a single microfluidic cartridge. Alternatively, a simplified cooling module can be obtained by connecting the cooling panels in series. An example of such a configuration can be seen in FIGS. 7A to 7C.

FIGS. 7A and 7B show different orientations of an alternative cooling module in the form of cold plate 736, and FIG. 7C shows an example microfluidic cartridge 701 in position on cold plate 736. FIG. 7A shows cold plate 736 from above, with coolant inlet connector 738 and coolant outlet connector 740 extending from the lower surface of cold plate 736. Cold plate 736 is provided with two cooling panels 742 and 744, each configured to cool a row or group of four thermocycling chambers on microfluidic cartridge 701 as shown in FIG. 7C. However, other configurations of cold plate and cooling panel are possible, depending on the configuration of the microfluidic cartridge. Cooling panel 742 and cooling panel 744 are each provided with a plurality of cooling fins 746, which act as heat sinks to absorb latent heat from microfluidic cartridge 701, more particularly from a group of thermocycling chambers present on microfluidic cartridge 701. Cooling fins 746 may be formed of any thermally conductive material, for example a material selected from aluminium, copper, brass, or aluminium nitride, and are in thermal contact not only with microfluidic cartridge 701 but also with a working coolant fluid flowing through cold plate 736. Good thermal contact between cooling fins 746 and microfluidic cartridge 701 can be obtained by using a compliant thermal interface material as described previously.

As can be seen from the illustrative arrows in FIG. 7A, the direction of working fluid flow through cooling panels 742 and 744 is the same, and that working coolant exits cooling panel 742 and enters cooling panel 744 from below the plane of the cooling panels. Thus, cooling panels 742 and 744 are connected in series, with working coolant exiting the second cooling panel (denoted 744) and exiting cold plate 736 via coolant outlet connector 740. As can be seen in FIG. 7B, a coolant channel 748 is provided on the underside of cold plate 736, thus enabling the transport of working coolant from the first cooling panel 742 to second cooling panel 744. In this way, a simplified coolant circuit on cold plate 736 can be realized. In other configurations, cooling panels 742 and 744 may be connected to a coolant source in parallel. Although not shown, cold plate 736 can be connected via coolant inlet connector 738 and coolant outlet connector 740 to a refrigeration module, which may include a pump to circulate working coolant through cold plate 736 and a radiator module to effect heat transfer from the warmed working coolant exiting cold plate 736 so that the latent heat is absorbed and the working coolant is cooled prior to re-entering cold plate 736. The refrigeration module may be capable of providing up to 750 W of cooling at 25° C. depending on coolant used, with feedback control enabled by inclusion of a first thermistor 750a coupled to coolant inlet connector 738 and a second thermistor 750b coupled to coolant outlet connector 740. Refrigeration modules and coolants are commercially available, such as the Koolance ALX-750-P400 system, and associated Koolance 702 Liquid Coolant, which is compatible with a wide range of metallic and thermoplastic materials.

In use, cooling module 103 may be operated in thermal contact with microfluidic cartridge 101 and at a constant temperature of no more than 35° C., for example no more than about 30° C., to ensure cooling rates of 30° C. per second within thermocycling chamber 106. Achieving such temperatures may include flowing water, propylene glycol or any other suitable coolant or working fluid through the cooling module at a rate of from 1 L/min up to 5 L/min, for example from 1.1 L/min to 4 L/min, for example from 1.3 L/min to 3.5 L/min, for example from 1.5 L/min to 3.3 L/min, for example from 2 L/min to 3 L/min.

### Optical Sensor

The PCR system 100 of FIG. 1 comprises an optical sensor 105 configured to obtain optical signals from the thermocycling chamber where thermocycling is performed. In some examples, the optical sensor 105 is a fluorescence sensor and the optical signals are fluorescence signals. As described above, fluorescent molecules are used as reporter molecules in PCR amplification, with the fluorescence intensity proportional to the amount of amplified nucleic acid material. In some examples, optical sensor 105 comprises a light source and a detector, wherein the light source is for example a laser diode, or an LED, configured to emit light of a wavelength suitable to cause fluorescence of a fluorescent reporter molecule during a PCR amplification process. For example, SYBR Green I, absorbs blue light with a λₘₐₓ of 497 nm, and emits green light with a λₘₐₓ of 520 nm. In some examples, the detector may be a charge coupled device (CCD) or pin photodiode configured to detect the emitted fluorescent light. In some examples, the detector may be a charge coupled device (CCD) or pin photodiode to detect the emitted fluorescent light. In some examples, optical sensor 105 is arranged above or below the thermocycling chamber, for example above or below a plane in which the liquid sample is being thermocycled. In some examples, microfluidic cartridge 101 is provided with an optical window or opening that allows transmission of light therethrough to optical sensor 105 located in PCR system 100 but external to microfluidic cartridge 101, or within microfluidic cartridge 101 itself. In some examples, optical sensor 105 is embedded into a lid of microfluidic cartridge 101.

The optical sensor may be configured to emit and receive optical signals to a pre-determined location in the thermocycling chamber. For example, the optical sensor may be configured to irradiate a region of the thermocycling chamber having a largest dimension (for example a diameter) of from 0.5 to 3 mm, for example from 1 to 2 mm, and to receive signals, for example fluorescence, from that same region. Since the thermocycling chamber can have a total surface area much greater than this, multiplexed PCR within the same chamber using immobilized reagents and zero fluid flow as described herein becomes possible, with multiple fluorescence detectors each focussed on a particular region of the thermocycling chamber.

### Other Components

In some examples, PCR system 100 may be provided with a magnet in or under microfluidic cartridge 101. In some examples, the magnet comprises a permanent magnet or an electromagnet. When used in combination with one or more magnetic beads in a PCR reaction mixture, with an oligonucleotide primer of an oligonucleotide primer pair linked to a magnetic bead, the magnet can draw the bead to the surface of the thermocycling chamber and thus bring the oligonucleotide primer (and target nucleic acid bound to the primer through Watson-Crick base-pairing) into close proximity to the heater, and, if present, a second oligonucleotide primer lyophilised on the surface. In this way, any possible diffusion of the oligonucleotides can be further limited. A cleaving agent may then be used to cleave the oligonucleotide primer from the bead to avoid any steric interference by the bead in the amplification reaction. Thus, in some examples, a cleaving reagent may also disposed with the lyophilized primer. In some examples, one or both of a cleaving reagent and a degrading enzyme is disposed with the lyophilized primer. The cleaving reagent may be in solution with the primer when pipetted onto the surface or may be added as part of a separate solution which is then freeze-dried as described. The cleaving reagent may be deposited on top of, or adjacent to the reaction reagent.

The nature of the cleaving reagent will depend on the initial functionalisation of the bead that allows covalent attachment of the oligonucleotide. Linker groups can be attached to the bead and the oligonucleotide may be covalently bound to the linker group. In some examples, the oligonucleotide is bound to the bead via a short peptidic linkage which can be cleaved enzymatically. For example, cathepsin B is a protease that cleaves a peptide bond at the C-terminal side of a dipeptide such as Phe-Arg bound to another moiety. Other enzyme-cleavable linkers can be based on β-galactoside, which can be degraded using β-galactosidase. This use of the bead is discussed further in this application in connection with the PCR method.

In some examples, PCR system 100 comprises an electrical interface, configured to contact an electrical interface provided on microfluidic cartridge 101. The electrical interface on microfluidic cartridge 101 may be coupled to any component of the cartridge that requires electrical current to operate. Examples of such devices include the heater elements, either in flat panel form or printed conductive trace form, and actuators for controlling fluid flow within the microfluidic cartridge. In some examples, the electrical interfaces may be multi-pin input/output off board connecters, for example 44-pin connectors that enable electrical coupling of the microfluidic cartridge to a computer module of the PCR system. Each pin of the electrical interface may provide an electrical contact to a specific component of the microfluidic cartridge, such as the individually addressable or controllable heaters described herein. The electrical coupling of the cartridge to the system allows control signals from the computer module to be sent to the cartridge so that electrical current can be sent to desired modules of the cartridge.

As noted above, PCR system 100 may comprise a computer control module. In some examples, the computer control module comprises a processor comprising hardware architecture to retrieve executable code from a data storage device or computer-readable medium and execute instructions in the form of the executable code. The processor may include a number of processor cores, an application specific integrated circuit (ASIC), field programmable gate array (FPGA) or other hardware structure to perform the functions disclosed herein. The executable code may, when executed by the processor, cause the processor to implement the functionality of one or more hardware components of the cartridge and/or system such as one or more heaters and/or one or more optical detectors. In the course of executing code, the processor may receive input from and provide output to a number of the hardware components, directly or indirectly. The computer control module may communicate with such components via a communication interface which may comprise electrical contact pads, electrical sockets, electrical pins or other interface structures. In one example, the communication interface may facilitate wireless communication.

In some examples, the computer control module facilitates the introduction of a sample into the thermocycling chamber, or into multiple thermocycling chambers. For example, the computer control module may control a series of valves and pumps in the system or on the microfluidic cartridge to direct flow of a test sample or solution to the thermocycling chamber. Thus, the system may include one or more valves and pumps. The pumps may be any type of pump suited for microfluidic usage. Suitable pumps include pressure-driven fluid drivers, e.g. hydraulic pumps coupled to a flow channel inlet and which build back-pressure in a system to drive fluid through a channel. Thermally driven pumping systems can include a series of thermal actuators in or adjacent a flow channel of the microfluidic device and which can be actuated in turn, causing cavitation of air bubbles in a liquid which then collapse causing a liquid flow. Other types of pumps include membrane-based systems, in which a surface of a flow channel or the thermocycling chamber is formed of a flexible membrane, which can be distorted by, for example, hydraulic action, again causing liquid flow.

In some examples, the computer control module may further control the processing of a sample in a thermocycling chamber, for example by subjecting the thermocycling chamber to thermocycling conditions. For example, the computer control module may control, through the output of control signals, the operation of one or more heaters to control the temperature and duration of heating within the or each thermocycling chamber, or the operation of one or more valves or pumps within a cooling module to control the temperature of the cooling module and thereby provide cooling to a reaction mixture provided in the thermocycling chamber. As a result, a sample may undergo various selected reactions, various selected heating cycles and various sensing operations under the control of the computer control module.

### Method of Performing PCR

A method of performing PCR, comprising:
introducing a sample suspected of containing a nucleic acid of interest into a microfluidic cartridge of a PCR system, the PCR system having an optical sensor, and a cooling module in thermal contact with a surface of the microfluidic cartridge, wherein the microfluidic cartridge comprises a thermocycling chamber having a planar floor provided with a heater;
introducing the sample and a PCR Master Mix capable of amplifying the nucleic acid of interest into the thermocycling chamber, to form a reaction mixture in the thermocycling chamber;
subjecting the reaction mixture to thermocycling conditions suitable for amplification by polymerase chain reaction by providing heat from the heater to the reaction mixture and extracting heat from the reaction mixture to the cooling module; and
detecting an optical signal from the reaction mixture.

The method may be performed on a microfluidic device as described herein, or on a PCR system as described herein comprising the microfluidic device described herein.

A sample suspected of containing a nucleic acid of interest can into a microfluidic cartridge of a PCR system by any suitable means. For example, the sample may be directly introduced by manual injection using a pipette into a sample inlet port provided on the microfluidic cartridge, or indirectly by introducing the sample into a sample port of the PCR system which is external to the microfluidic cartridge, with the sample then being pumped into the microfluidic cartridge.

A PCR “Master Mix” is introduced into the thermocycling chamber. A PCR Master Mix is an aqueous solution of PCR reagents, already at optimized concentrations, which can be readily aliquoted and added to the reaction mixture. The Master Mix usually comprises the DNA elongation enzyme (e.g. a polymerase), the dNTPs, MgCl₂ as an enzyme co-factor (although other co-factors, such as MgSO₄ may be used with certain enzymes), all dissolved in an aqueous buffer. Suitable polymerases include the thermostable polymerases Taq, Bst and Pfu. The Master Mix may also include a reporter molecule, such as a non-specific fluorescent dye, such as SYBR Green, which intercalate into any double-stranded DNA, leading to an increase in fluorescence as more double-stranded DNA is produced, while other suitable reporter molecules include target-specific fluorescent reporter molecules, such as the TaqMan hydrolysis probes. The reporter molecule may be dissolved in the Master Mix, or may be covalently bound to a primer. The LightCycler® 480 SYBR Green I Master Mix includes a polymerase, co-factor, dNTPs and SYBR Green I in a buffered solution, meaning that only the nucleic acid sample (and, if appropriate, a primer) need to be added. However, the reporter molecule may also be added separately.

Similarly to the sample, a PCR Master Mix may be introduced into the thermocycling chamber in a number of ways. As described above, a reagent of the PCR Master Mix, for example an oligonucleotide primer may be disposed on a surface of the thermocycling chamber, with or without being isolated using a dissolvable or degradable film, with the remaining components of the PCR Master Mix being introduced separately or together, in solution. The PCR Master Mix may be introduced into a reservoir, with aliquots being pumped to one or more thermocycling chambers, or may be introduced directly into a thermocycling chamber. The PCR Master Mix may be provided in a frangible package, for example a blister pack, which can be ruptured to release the PCR Master Mix into a reagent reservoir from which it can be pumped into a thermocycling chamber.

In some examples, the PCR Master Mix comprises at least one magnetic bead and one oligonucleotide of the oligonucleotide pair is attached to the at least one magnetic bead. A magnet can then be used to draw the at least one magnetic bead to the inner surface of the thermocycling chamber, for example to a location at which the second oligonucleotide of the oligonucleotide pair is disposed. Since the oligonucleotide primer bound to the magnetic bead is complementary to the target nucleic acid of interest, the target (if present) can anneal to that oligonucleotide and also be brought to the surface of the thermocycling chamber by the magnetic bead. In this way, the bound oligonucleotide primer and target nucleic acid are brought into close proximity to the oligonucleotide primer immobilized on the surface. In some examples, the magnetic beads comprise of an iron oxide core, and a polymer coating. The surface of the polymer coating may also comprise functional groups which may then be covalently linked to a primer. In some examples, the bead is a colloidal magnetite (Fe₃O₄), maghemite (Fe₂O₃) or ferrite which has been surface-modified by silanisation. In some examples, the bead particle comprises a polymer core (for example polystyrene), a metal oxide shell (for example iron oxide) and a polymer coating. Examples of magnetic beads that can be covalently linked to an oligonucleotide primer include Dynabeads® from ThermoFisher.

In some examples, a reaction mixture may be prepared by combining the nucleic acid sample and a PCR Master Mix. In some examples, the reaction mixture may be prepared by combining the nucleic acid sample, an oligonucleotide which is covalently bound to a magnetic bead, the dNTPs, polymerase and buffer/salts, and heating the reaction mixture to denature any double stranded DNA in the nucleic acid sample and hybridise the oligonucleotide (covalently bound to the magnetic bead) to its complementary target nucleic acid of interest, if the target is present in the sample. In this way, not only is the oligonucleotide primer brought to the surface of the thermocycling chamber by the magnet, but also the target nucleic acid sequence of interest. The magnetic bead limits diffusion of the oligonucleotide primer, and a nucleic acid hybridised or annealed to the oligonucleotide primer.

In some examples, the inner surface of the thermocycling chamber comprises a plurality of first oligonucleotides of a plurality of oligonucleotide pairs, each complementary to a different nucleic acid of interest, with each of the first oligonucleotides at a discrete, spaced apart location, and wherein the method comprises performing multiple nucleic acid amplification reactions in parallel. Accordingly, a plurality of second oligonucleotides of the plurality of the oligonucleotide pairs may be required. In these examples, which enable a multiplexed PCR analysis, each corresponding second oligonucleotide may be bound to a separate magnetic bead as described. Alternatively, the second oligonucleotide or the plurality of second oligonucleotides may be disposed with their respective first oligonucleotides and isolated by the thermally dissolvable or degradable film. Thus, references to the first oligonucleotide being exposed to a reaction mixture refer also to the second oligonucleotide being exposed to the reaction mixture, when that is also isolated under the film. It is also possible for the second oligonucleotide to be present in a reaction mixture without being bound to a magnetic bead, or to be introduced into the reaction chamber separately to the reaction mixture (whether bound to a magnetic bead or not).

In addition to multiplexed PCR reactions within a single chamber, multiple primer pairs may be included in a reaction mixture to perform nested PCR amplification, to address the issue of non-specific primer annealing at unexpected binder sites. A reaction mixture for nested PCR amplification can include a first primer pair, designed to amplify a target nucleic acid sequence which includes a sequence of interest flanked at each end by an additional sequence, and a second primer pair designed to only bind to the sequence of interest within the amplification product from an amplification using the first primer pair.

During the method, a reaction mixture comprising a sample and a PCR Master Mix may be flowed into a thermocycling chamber or into each one of multiple thermocycling chambers. The sample may also be introduced into the thermocycling chamber separately to a PCR Master Mix, with these being combined and mixed in the thermocycling chamber to form a reaction mixture in situ. In some examples, once the reaction mixture has completely filled the thermocycling chamber to the exclusion of any air bubbles which can be expelled via a vent, no further fluid flow occurs in the thermocycling chamber. That is, in some examples, once a sample and PCR Master Mix have been introduced into the thermocycling chamber, subjecting the reaction mixture to conditions suitable for amplification by polymerase chain reaction comprises thermocycling in the absence of fluid flow within the thermocycling chamber

In examples in which one or more reagents are immobilized by a thermally dissolvable or degradable film in the thermocycling chamber, once the sample and Master Mix have been introduced into the thermocycling chamber the at least one inner surface of the thermocycling chamber may then be heated (for example by providing a current to a PCB forming at least part of the substrate) to a temperature which causes the thermally dissolvable or degradable film to dissolve and/or degrade as described, so that the reagent, e.g. an oligonucleotide primer, can be exposed. In some examples, the temperature to which the solution is to be heated may depend upon the composition of the film.

In some examples, the temperature to which the solution is to be heated may be from 40 to 120° C., for example from 50 to 110° C., for example from 60 to 100° C., for example from 70 to 90° C. In some examples, the temperature may be from 90° C. to 100° C. An immobilized reagent such as an oligonucleotide primer will then be exposed, and may then solubilise into the solution in the same location where it was originally disposed. As there is minimum or zero flow in the thermocycling chamber, the motion of the nucleic acid material (the two oligonucleotides serving as primers, and the larger nucleic acid of interest) is limited because of diffusion.

In some examples, the inner surface of the thermocycling chamber comprises a plurality of first oligonucleotides of the plurality of oligonucleotide pairs, each at a discrete, spaced apart location. In some examples, each oligonucleotide pair is complementary to a different nucleic acid of interest. As different locations in the thermocycling chamber comprise different oligonucleotides which can act as primers for different target nucleic acids of interest, simultaneous tests for the presence of different targets can occur. In some examples, the individual locations are spaced apart to avoid cross-contamination. In some examples, the individual locations are spaced from 100 μm to 1000 μm apart, in some examples from 200 to 800 μm apart, in some examples 300 to 600 μm apart, and in some examples 500 μm apart.

The first oligonucleotide or each first oligonucleotide of a plurality of oligonucleotide pairs may be immobilized on the inner surface of the thermocycling chamber, remaining immobilized after a thermally dissolvable or degradable film has been removed. Immobilizing the oligonucleotide ensures that there is no diffusion whatsoever and constrains the reaction to that location. However, to ensure that the PCR reaction is not impeded by the surface, a second oligonucleotide or each second oligonucleotide of a plurality of oligonucleotide pairs may be in solution, and not bound to a magnetic bead.

In some examples, one or both of a degrading enzyme and a cleaving reagent is also isolated with a primer of a primer pair by the thermally dissolvable or degradable film and wherein, after the heating step, the cleaving reagent is released and cleaves the second primer of the primer pair from the at least one bead and/or the degrading enzyme is released and degrades the film. In some examples, a cleaving reagent is also disposed along with the first primer as described herein. Upon softening, or dissolving, of the thermally dissolvable or degradable film, the cleaving reagent may also be released. This cleaving reagent may then cleave the second primer from the bead, allowing the PCR reaction to occur in solution and thus be more efficient than a surface-based reaction. In some examples, the second oligonucleotide may be cleaved using external influence, for example, using heat or UV light, instead of enzymatically. UV-cleavable linkers include the nitrobenzyl linker.

In some examples, a reaction mixture comprising sample and PCR Master Mix has a volume of less than 100 μL, for example less than 50 μL, for example less than 25 μL, for example less than 10 μL, for example about 5 μL. In some examples, the reaction mixture has a volume of greater than 5 μL, for example greater than 10 μL, for example greater than 25 μL, for example greater than 50 μL, for example about 100 μL.

In some examples, a sample or reaction mixture comprises a nucleic acid sample obtained from a subject. In some examples, the nucleic acid sample may comprise a nucleic acid for analysis and is to be amplified in a method as described herein. In some examples, the nucleic acid sample may comprise a plurality of nucleic acids for analysis which are to be amplified in a method as described herein. In some examples, the reaction mixture is suspected of comprising a one or a plurality of nucleic acid sequences of interest. In some examples, the nucleic acid sample is obtained from one or more of a blood sample, a tissue sample, a saliva sample or mucosal sample. In some examples, the nucleic acid sample is obtained using a swab. In some examples, the nucleic acid sample is isolated from the bodily fluid or tissue via which it was obtained. Isolating the nucleic acid ensures that no other component of the sample is present which could inhibit PCR amplification. In some examples, the nucleic acid sample is not isolated from the bodily fluid or tissue via which it was obtained. In some examples, the nucleic acid sample obtained from a subject is incorporated into a reaction mixture with or without any isolation or preparation. In some examples, the nucleic acid sample obtained from a subject is dissolved or dispersed in an aqueous solution, thus forming a reaction mixture.

In some examples, a second oligonucleotide of an oligonucleotide pair complementary to a nucleic acid of interest which is suspected of being in the nucleic acid sample is present in the reaction mixture when the nucleic acid sample is dissolved or dispersed in the solution, or is added to the solution after the nucleic acid sample has been dissolved or dispersed. The second oligonucleotide may be dissolved or suspended in the reaction mixture before or after the nucleic acid sample has been dissolved or dispersed, or the second oligonucleotide may be mixed with the nucleic acid sample before being added to the reaction mixture. In some examples, a second oligonucleotide of an oligonucleotide pair complementary to a nucleic acid of interest which is suspected of being in the nucleic acid sample is introduced into the reaction chamber of the microfluidic device separately to the reaction mixture. For example, the second oligonucleotide of the oligonucleotide pair may also be disposed on the inner surface of the reaction chamber and be isolated under the thermally dissolvable or degradable film, or it may be introduced as a separate solution before or after the reaction mixture has been introduced.

In some examples, a portion of oil is provided into the thermocycling chamber. The function of the oil is to prevent evaporation of any liquid from the liquid medium during amplification. In some examples, light mineral oils such as a white oil are provided. In some examples, the volume of oil provided is in excess of the volume of the liquid medium. In some examples, sufficient oil is provided to completely envelop the liquid medium.

Once the liquid volume has been introduced into the thermocycling chamber, heat is provided to raise the temperature of the reaction mixture. In some examples, heat is provided by means of a heater in the form of a printed electrical trace provided in or on the substrate on which the thermocycling chamber is located.

Since the microfluidic cartridge is in thermal contact with the cooling module when present in the PCR system, when the temperature of the reaction mixture needs to be adjusted, the cooling module, which is maintained at a temperature of no more than about 35° C., for example about 25° C., can extract heat from the reaction mixture.

In some examples, the cooling module includes a working fluid or coolant, and the fluid is flowed through the cooling module to provide cooling and extract heat from the reaction mixture. In some examples, the fluid is selected from water, ethylene glycol and propylene glycol, and mixtures thereof, and is flowed through the cooling module at a flow rate of up to 4 L/min, for example from 1 L/min up to 4 L/min, for example from 1.1 L/min to 4 L/min, for example from 1.3 L/min to 3.5 L/min, for example from 1.5 L/min to 3.3 L/min, for example from 2 L/min to 3 L/min. While greater, or quicker, heat transfer may be achieved using a flow of working fluid through the cooling module, adequate heat transfer may also be achieved without any fluid flow. Thus, in some examples, the fluid is static in the cooling module.

In some examples, the method comprises performing one or more rounds of PCR amplification with the reaction mixture prior to any optical detection such as fluorescence detection.

In a situation in which a target nucleic acid in a sample is RNA, rather than DNA, then the sample may initially be subjected to conditions suitable for reverse transcription, to convert the RNA to cDNA (“copy DNA”). In this situation, the PCR Master Mix will include a reverse transcriptase, or the reverse transcriptase may be immobiilzed on a surface of the thermocycling chamber as described previously in connection with the PCR primers. Regardless of how the reverse transcriptase is introduced, the reaction mixture is heated to a temperature in the range of from 25° C. to 70° C., for example in the range of from 35° C. to 60° C., for example in the range of from 40° C. to 50° C. After the RNA has been reverse-transcribed into cDNA, the nucleic acid is subjected to amplification conditions by PCR using a polymerase.

The three basic steps of a single round of PCR amplification are denaturation, annealing and chain extension, each optimally taking place at different temperatures (for example in the range of 94-98° C. for denaturation; 50-65° C. for annealing, and 70-80° C. for chain extension, depending on polymerase). A reaction mixture present in the thermocycling chamber of the microfluidic device described herein may therefore be heated by the heater in the microfluidic device to a denaturation temperature of from 94-98° C. for a sufficient time for any double stranded DNA to separate or denature into single stranded DNA. According to the thermocycling protocol described above, the reaction mixture must then be cooled to an annealing temperature of from 50-65° C. While the turning off of a heater in thermal contact with the reaction mixture will cease any further heating, it will not rapidly cool the reaction mixture, as is desirable. However, with the introduction of the cooling module as described herein, heat can be dissipated away from the reaction mixture to a fluid (for example water) flowing through the cooling module.

For each cycle of amplification, the duration of the denaturation step may account for 10-20% of the cycle duration, while the annealing step may account for 10-30% of the cycle duration and the extension step may account for 40-80% of the cycle duration. In some examples, the denaturation step may take from 0.1 to 3 seconds, and in some examples, 0.5 seconds. In some examples, the annealing step may take from 0.1 to 3 seconds, and in some examples, 0.5 seconds. In some examples the extension step may take from 1 to 60 seconds, and in some examples, from 5 to 10 seconds. In some examples, the nucleic acid is subjected to amplification conditions by PCR by thermocycling the reaction mixture for up to 50 cycles, for example from 10 to 50 cycles, or from 20 to 50 cycles, or about 40 cycles.

The conditions suitable for amplification by polymerase chain reaction may comprise providing heat from the heater to the thermocycling chamber to heat the reaction mixture at a heating rate of from 20° C./second to 200° C./second. The conditions suitable for amplification by polymerase chain reaction may comprise heating the reaction mixture to a denaturing temperature of the nucleic acid of interest by providing a pulse of energy to a heater of the microfluidic device. The conditions suitable for amplification by polymerase chain reaction may comprise pulse-controlled amplification, in which a time varying heat flux is applied to the reaction mixture resulting in a time varying temperature gradient in the reaction mixture, as will be explained in more detail below.

While each of the temperatures associated with the three stages of PCR may be considered as a step of a thermocycle, each thermocycle necessarily also involves a heating step which begins before the denaturing step, but which may also overlap with the denaturing step. In this heating step, the temperature of the reaction mixture with respect to the starting temperature or the temperature of a previous elongation step is increased at least locally, e.g. at the surface of the thermocycling chamber adjacent the heater, where magnetic beads and reagents are concentrated, in order to facilitate denaturing. Subjecting the reaction mixture to conditions suitable for amplification by polymerase chain reaction may therefore comprise heating the reaction mixture to a denaturing temperature of the nucleic acid of interest by providing a pulse of energy to an embedded heater. Through the effect of the heater, a time varying heat flux is applied to the reaction mixture, resulting in a time varying temperature gradient in the reaction mixture, meaning a localised temperature of at least 90° C., for example at least 95° C., can rapidly be reached in the vicinity of the heater. This pulsing of energy can be used in conjunction with immobilized reagents as described above, as the reagents are in close proximity to the embedded heater and so are affected by the localised temperature increase more than if the reagents were in free solution and further away from the surface with the embedded heater.

The length of time of heating is the total duration, in which pulses of energy are provided to the heater for it to transmit heat with a power suitable for denaturing of a nucleic acid sample, which may correspond to a transient, localised heating of the reaction mixture to a temperature of at least 90° C. The heating time is the total duration, in which pulses of energy are provided to the heater so that heat flows from the heater to the reaction mixture to bring about a temperature increase that is suitable for denaturing.

The unravelling of a DNA double strand and diffusion of the two strands from one another (to avoid re-hybridization), can require a sufficiently high temperature to be maintained for a certain period of time. However, since the heater effects a localised heating, the duration of pulsed heating to effect denaturation may be less than 10 seconds, for example less than 5 seconds, for example less than 3 second, for example less than 1 second, for example less than 500 ms (milliseconds), for example less than 250 ms, for example less than 100 ms, for example less than 50 ms, for example less than 25 ms, for example less than 10 ms, for example less than 8 ms, for example less 3 ms, for example less than 1 ms, for example less than 500 μs (microseconds), for example less 300 μs, for example less than 100 μs, for example less than 50 μs, for example less than 30 μs, for example less than 10 μs.

For example, for a volume of 30 μL in a thermocycling chamber, increasing the temperature of an aqueous solution by up by 30° C. (as would be required to take a test solution from a chain elongation temperature of 70-80° C. to a denaturation temperature of 90-95° C.) can be achieved by heating a single serpentine heater of etched copper with a pulse of energy having a heat flux of 4000 kW/m² for 1 ms (millisecond), with a pulse of energy having a heat flux of 400 kW/m² for 10 ms (milliseconds), or with a pulse of energy having a heat flux of 40 kW/m² for 100 ms (milliseconds). In another example, heating a single serpentine heater of etched copper having a footprint of 450 mm² by providing 115 W of DC power at a duty of 40% for less than, for example, 250 milliseconds as described above provides for a heating ramp rate of 140° C./second to a denaturing temperature of 95° C., while a duty of 25% just as rapidly heats a liquid in the thermocycling chamber to an annealing temperature of 55° C. Thus, the heater is able to rapidly heat a small interfacial volume of reaction mixture in the thermocycling chamber and rapidly and reliably effect denaturation.

As described earlier, the heater of the microfluidic cartridge may include a plurality of etched copper serpentine heaters, each provided in a different layer or plane of a substrate underlying the reaction chamber. For such a system, increasing the temperature of a reaction mixture from a chain elongation temperature of 70-80° C. to a denaturation temperature of 90-95° C. can be achieved by heating the heaters with a pulse of energy having a combined heat flux of 4000 kW/m² for 1 ms (millisecond), with a pulse of energy having a combined heat flux of 400 kW/m² for 10 ms (milliseconds), or with a pulse of energy having a combined heat flux of 40 kW/m² for 100 ms (milliseconds).

As described earlier, the heater may include an embedded heater such as an etched copper trace overlaid with a diffuser layer or heat spreader of thermally conductive material. For such a system, increasing the temperature of a reaction mixture from a chain elongation temperature of 70-80° C. to a denaturation temperature of 90-95° C. can be achieved by heating the heater with a pulse of energy with a heat flux of 4000 kW/m² for 1 ms (millisecond), a pulse of energy with a heat flux of 400 kW/m² for 1ms (millisecond), or pulse of energy with a with a heat flux of 400 kW/m² for 10 ms (milliseconds), and allowing the diffuser layer to diffuse the generated heat into the thermocycling chamber.

Exact power requirements to bring a reaction mixture to an annealing temperature may vary for any given system. For example, required power input will depend on the dimensions of the heater, with required power being scalable with heater area, but may also depend on the presence or absence of a compliant thermal interface material, and whether or not the embedded heater is embedded in the substrate or fluidic stack so as to form an internal surface of the thermocycling chamber, or is fully embedded within a substrate layer.

Once denaturation has been effected, a cooling step begins before the annealing step, in order to reach the temperature required for annealing, usually 50-65° C. The cooling may take place through heat transfer to the cooling module, once power to the heater has been stopped. Since the cooling module is maintained at a temperature below the thermocycling temperatures, for example 35° C. or less, for example about 25° C., then rapid cooling rates of from 20° C./second to 100° C./second, for example about 30° C./second can be obtained.

Thus, the duration of cooling to effect annealing of the primers to the nucleic acid at the predetermined location may be less than 10 seconds, for example less than 5 seconds, for example less than 3 second, for example less than 1 second, for example less than 500 ms (milliseconds), for example less than 250 ms, for example less than 100 ms, for example less than 50 ms, for example less than 25 ms, for example less than 10 ms, for example less than 8 ms, for example less 3 ms, for example less than 1 ms, for example less than 500 μs (microseconds), for example less than 300 μs, for example less than 100 μs, for example less than 50 μs, for example less than 30 μs, for example less than 10 μs.

Once annealing has been effected, a heating step begins before the chain extension or elongation step, in order to reach the temperature required for chain extension, usually 70-80° C.

The length of time of heating for chain extension is the total duration, in which the heater transmits heat with a power suitable for chain extension of a nucleic acid sample, which may correspond to a transient, localised heating of the reaction mixture to a temperature of about 70-80° C. The heating time is the total duration, in which heat flows from the heater to the reaction mixture to bring about a temperature increase suitable for chain extension.

In one example, the annealing temperature is equal to the chain extension temperature. If the annealing temperature is equal to the chain extension temperature, only one temperature cycle with two different temperatures is required to amplify the nucleic acid of interest. The melt temperatures of the primers and the polymerase used may be selected so that at the primer melting temperature the polymerase used can still synthesize DNA at a sufficient speed. In this example, the temperature for annealing and chain extension is achieved by global heating and the denaturing step is achieved through localised heating at the predetermined location.

The duration of heating to effect chain extension may be less than 10 seconds, for example less than 5 seconds, for example less than 3 second, for example less than 1 second, for example less than 500 ms (milliseconds), for example less than 250 ms, for example less than 100 ms, for example less than 50 ms, for example less than 25 ms, for example less than 10 ms, for example less than 8 ms, for example less 3 ms, for example less than 1 ms, for example less than 500 μs (microseconds), for example less 300 μs, for example less than 100 μs, for example less than 50 μs, for example less than 30 μs, for example less than 10 μs.

In one example, the heater comprises an array of 75 gold-coated tungsten wires (15 μm diameter, 200 nm Au coating) arranged on an internal surface of the thermocycling chamber. Localised heating of a layer of reaction mixture of a few micrometers depth adjacent the wire is achieved via application of sub-millisecond voltage pulses to the wires. In particular, for providing localized heating for a chain extension step, a pulse at substantial peak power in the order of 1 KW for less than 500 microseconds is applied to the wire array from a 10 mF capacitator loaded to 30-40 V via a MOSFET (metal-oxide-semiconductor field-effect transistor; serving as a fast switch). This is sufficient to locally heat the solution to 60° C. to 80° C. for chain extension to occur.

In another example, localized heating for a chain extension step can be achieved by heating a thin film flat panel heater (tungsten film, 15 μm thickness with a 200 nm thick Au coating) with a heat flux of 4000 kW/m² for 1 ms (millisecond), with a heat flux of 400 kW/m² for 10 ms (milliseconds), or with a heat flux of 40 kW/m² for 100ms (milliseconds) from a 10 mF capacitator loaded to 30-40 V via a MOSFET (metal-oxide-semiconductor field-effect transistor.

Thus, an embedded heater as described herein is able to rapidly heat a small volume of reaction mixture in the thermocycling chamber and rapidly effect chain extension of the primers by the polymerase, using a nucleic acid of interest as the template.

One example of a thermocycling protocol may include:
introducing a sample suspected of containing a nucleic acid of interest into a microfluidic cartridge of a PCR system as described herein;
introducing a PCT Master Mix into the microfluidic cartridge;
pumping the sample and PCR Master Mix into the thermocycling chamber, to form a reaction mixture in the thermocycling chamber;
operating the cooling module so as to maintain a substantially constant temperature of about 25° C.;
providing power to the heater to heat the reaction mixture to a temperature of 90° C. to 100° C. at a ramp rate of 20° C./second to 200° C./second to allow denaturation to occur;
switching off power to the heater and actively cooling the reaction mixture using the cooling module at a cooling rate of 20° C./second to 100° C./second to cool the reaction mixture to a temperature of 40° C. to 65° C. for annealing of the PCR primers to a target nucleic acid (if present in the sample);
providing power to the heater to heat the reaction mixture to a temperature of 60° C. to 80° C. at a ramp rate of 20° C./second to 100° C./second to allow chain extension to occur; and
operating a fluorescence detector to obtain fluorescence signals from the reaction mixture.

The fluorescence detector can be used to detect and measure the fluorescence level after each thermocycle, or after 5 thermocycles, or after 10 thermocycles, or any number of cycles as required. In other words, the fluorescence detection may be continuous detection, i.e. during and after each amplification cycle, or it may be an end-point detection after a pre-determined number of cycles. If a nucleic acid of interest is present in the sample, it will be amplified through the thermocycling, using the complementary oligonucleotide primer pair. If one primer of a primer pair is disposed on a surface of the thermocycling chamber at a particular location, and magnets and magnetic beads are used to bring the second primer and target nucleic acid (if present) to that location, measurement of any presence or increase in fluorescence at that location is an indication that the nucleic acid of interest was present in the sample or reaction mixture. The sooner that a positive result (via fluorescence detection) confirms that a nucleic acid of interest is present in a reaction mixture, the quicker the overall test time.

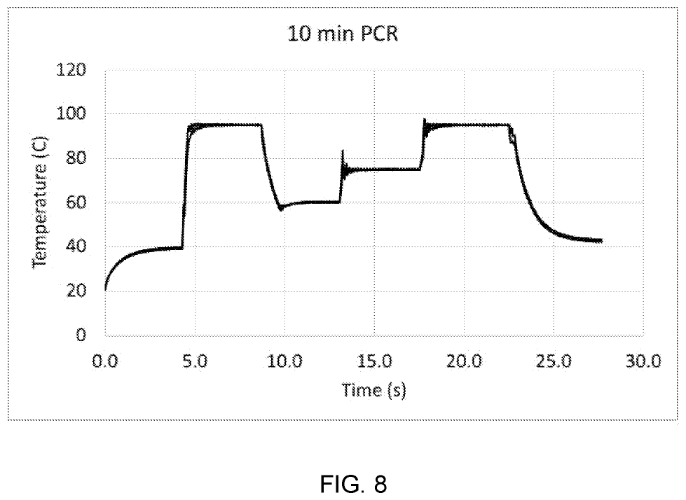

Through the use of a PCR system as described herein, a more rapid and energy efficient PCR method can be realised. As a proof-of-concept, a PCR system in accordance with the present disclosure was constructed, using the Koolance ALX-750-P400 cooling system, and associated Koolance 702 Liquid Coolant operating at a temperature of 30° C. and a flow rate of 2.1 L/min, and a microfluidic cartridge in thermal contact with the cooling module. The microfluidic cartridge was constructed using a PCB substrate with two etched copper serpentine heaters, one embedded in the substrate and the other being present on an upper surface of the substrate so as to be present on an inner surface of a thermocycling chamber formed on the PCB substrate. Each heater had an area of 15 mm×30 mm, and the thermocycling chamber was formed in a layer of pressure sensitive adhesive so as to have a chamber volume of approximately 20 μL and a chamber height of 150 μm as a base layer of a microfluidic stack. Electrical connections were provided to the heater, and the heater was heated by input of DC power of 115 W. At a duty cycle of 40%, an aqueous solution in the chamber was heated to a typical annealing temperature of 95° C. at a ramp rate of approximately 160° C./second. Turning the heater off enabled the cooling module to extract heat from the system at a cooling rate of almost 30° C./second. FIG. 8 shows the experimentally measured temperature profile of each heater as a function of time elapsed for a single PCR cycle, showing extremely close correlation with the two heaters, and the rapid heating and cooling rates. As can be seen from FIG. 8, one complete round of amplification takes less than 30 seconds, meaning a PCR amplification of 20 cycles, which is more than enough to obtain a definitive result even with low copy numbers of target, can be achieved in only 10 minutes.

While the systems, methods and related aspects have been described with reference to certain examples, it will be appreciated that various modifications, changes, omissions, and substitutions can be made without departing from the spirit of the disclosure. It is intended, therefore, that systems, methods and related aspects be limited only by the scope of the following claims. Unless otherwise stated, the features of any dependent claim can be combined with the features of any of the other dependent claims, and any other independent claim.

1. A PCR system, comprising:
a microfluidic cartridge having a thermocycling chamber, wherein a floor of the thermocycling chamber is substantially planar and is provided with a heater;
a cooling module configured to engage with and be in thermal contact with a surface of the microfluidic cartridge; and
an optical sensor configured to obtain optical signals from the thermocycling chamber.

2. The PCR system of claim 1, wherein an aspect ratio of the largest dimension of the thermocycling chamber to the chamber height is in the range of from 10:1 to 1000:1.

3. The PCR system of claim 1, wherein the heater comprises at least partially overlapping conductive traces provided in different layers of the microfluidic cartridge.

4. The PCR system of claim 1, wherein the heater comprises a series of electrical traces in a first layer of the microfluidic cartridge overlaid with a diffuser of thermally conductive material in a second layer of the microfluidic cartridge.

5. The PCR system of claim 1, wherein the heater comprises at least two perpendicularly oriented serpentine conductive traces provided in different layers of the microfluidic cartridge.

6. The PCR system of claim 1, wherein the microfluidic cartridge comprises a plurality of thermocycling chambers provided in at least two groups, and the cooling module comprises a number of cooling panels equivalent to the number of groups of thermocycling chambers.

7. The PCR system of claim 1, further comprising a layer of compliant thermal interface material between the cooling module and the microfluidic cartridge.

8. The PCR system of claim 1, wherein the thermocycling chamber is provided with a plurality of capillary breaks on an inner surface of the thermocycling chamber.

9. A method of performing PCR, comprising:
introducing a sample suspected of containing a nucleic acid of interest into a microfluidic cartridge of a PCR system, the PCR system having an optical sensor, and a cooling module in thermal contact with a surface of the microfluidic cartridge, wherein the microfluidic cartridge comprises a thermocycling chamber having a planar floor provided with a heater;
introducing the sample and a PCR Master Mix capable of amplifying the nucleic acid of interest into the thermocycling chamber, to form a reaction mixture in the thermocycling chamber;
subjecting the reaction mixture to thermocycling conditions suitable for amplification by polymerase chain reaction by providing heat from the heater to the reaction mixture and extracting heat from the reaction mixture to the cooling module; and
detecting an optical signal from the reaction mixture.

10. The method of claim 9, wherein the thermocycling conditions suitable for amplification by polymerase chain reaction comprise providing heat from the heater to the thermocycling chamber to heat the reaction mixture at a heating rate of from 20° C./second to 200° C./second.

11. The method of claim 9, comprising maintaining the cooling module at a constant temperature of no more than about 35° C.

12. The method of claim 9, comprising flowing a propylene glycol coolant through the cooling module at a rate of up to 4 L/min.

13. The method of claim 9, wherein one or more components of the PCR Master Mix are provided in lyophilised form on an internal surface of the thermocycling chamber.

14. The method of claim 9, wherein subjecting the reaction mixture to conditions suitable for amplification by polymerase chain reaction comprises thermocycling in the absence of fluid flow within the thermocycling chamber.

15. The method of claim 9, wherein subjecting the reaction mixture to conditions suitable for amplification by polymerase chain reaction comprises heating the reaction mixture to a denaturing temperature of the nucleic acid of interest by providing a pulse of energy to the heater.
